(19) United States
(12) Patent Application Publication
(10) Pub. No.: US 2022/0122695 A1
(43) Pub. Date: Apr. 21, 2022

(54) METHODS AND SYSTEMS FOR PROVIDING SAMPLE INFORMATION

(71) Applicant: IDBYDNA INC., San Francisco, CA (US)

(72) Inventors: Steven FLYGARE, Sandy, UT (US); Wan Rong XIE, San Francisco, CA (US); Hajime MATSUZAKI, Rancho Palo Verdes, CA (US); Brett HOUTZ, Hayward, CA (US); Robert SCHLABERG, Salt Lake City, CA (US); Qing LI, Mountain View, CA (US)

(21) Appl. No.: 17/290,734

(22) PCT Filed: Aug. 27, 2019
(86) PCT No.: PCT/US19/48363

§ 371 Date: Apr. 30, 2021

Related U.S. Application Data
(60) Provisional application No. 62/723,384, filed on Aug. 27, 2018.

(51) Int. Cl. G16B 30/10; G16B 45/00; C12Q 1/04; G16H 50/20; G16H 10/40

(52) U.S. Cl. CPC G16B 30/10; G16B 45/00; G16H 10/40; G16H 50/20; C12Q 1/04

(57) ABSTRACT

The present disclosure provides methods and systems for providing and/or displaying information corresponding to a sample. The information may comprise the identity of one or more microorganisms within the sample and may be based on an analysis of sequencing reads corresponding to the sample.

### CROSS-REFERENCE

This application claims priority to U.S. Provisional Patent Application No. 62/723,384 filed Aug. 27, 2018 which is entirely incorporated herein by reference.

### BACKGROUND

Samples may be analyzed for various purposes, including detecting the presence or amount of a target such as a nucleic acid molecule in a sample. Analysis of a sample comprising one or more nucleic acid molecules may involve sequencing the nucleic acid molecules, or portions or derivatives thereof. Sequencing may facilitate identification of contaminants and/or species of potential interest within a sample. For example, sequencing may be used to identify a microorganism or pathogen within a sample.

### SUMMARY

Recognized herein is a need to improve diagnostic testing for pathogens in patient samples. A diagnostic test may involve extracting ribonucleic acid (RNA) and deoxyribonucleic acid (DNA) molecules from a patient sample and preparing (e.g., independently preparing) sequencing libraries for both the RNA (e.g., RNA converted to complementary DNA (cDNA)) and DNA molecules. Molecular diagnostic tests using next generation sequencing (NGS) typically align reads to reference sequences using software such as BWA and display the aligned reads in a viewer such as the IGV. An alternative analysis is based on k-mers derived from reads and uses a classification algorithm to assign reads to organisms and place the reads within a reference genome or genes of interest. Results metrics such as k-mer uniqueness are specific to this analysis and require new ways to convey (e.g., visually convey) these values in the context of reviewing suspected pathogens in a patient sample. An interface useful for conveying such results may also support review of pathogens in the context of assessing sequencing quality control (QC), external processing controls, internal control organisms, and sample library quality that are specific to an infectious disease diagnostic test based on the analysis of the methods and systems described elsewhere herein.

Accordingly, the present disclosure provides methods and systems for providing information corresponding to a sample. In an aspect, the present disclosure provides a system for providing information corresponding to a sample, comprising a processor configured to display the information on a web-based graphical interface, wherein the information is represented by one or more visual and/or textual indicators, including (i) an entity indicator, and (ii) a quality control indicator, wherein the information comprises one or more identities of one or more entities associated with the sample, wherein the entity indicator provides information about the one or more identities of the one or more entities, and wherein the quality control indicator provides information about the certainty with which the one or more identities of the one or more entities are determined.

In some embodiments, an entity of the one or more entities is a human. In some embodiments, an entity of the one or more entities is a pathogen. In some embodiments, an entity of one or more entities is selected from the group consisting of a fungus, bacterium, parasite, and virus. In some embodiments, the one or more entities comprise a first entity that is a human and a second entity selected from the group consisting of a fungus, bacterium, parasite, and virus. In some embodiments, the second entity is associated with a disease or disorder. In some embodiments, the second entity is associated with an infection. In some embodiments, one or more additional entities are associated with a disease, disorder, or infection. In some embodiments, the one or more additional entities are selected from the group consisting of fungi, bacteria, parasites, and viruses. In some embodiments, the human has or is suspected of having a disease or disorder. In some embodiments, the human has been exposed or is suspected of having been exposed to a pathogen.

In some embodiments, the information represented by the entity indicator and the quality control indicator comprises data based on a plurality of sequencing reads corresponding to the one or more entities associated with the sample. In some embodiments, the plurality of sequencing reads comprises deoxyribonucleic acid (DNA) sequencing reads and/or ribonucleic acid (RNA) sequencing reads. In some embodiments, the plurality of sequencing reads comprise both DNA sequencing reads and RNA sequencing reads. In some embodiments, the plurality of sequencing reads is generated using sequencing by synthesis, sequencing by ligation, nanopore sequencing, or sequencing by hybridization. In some embodiments, the plurality of sequencing reads is generated using sequencing by synthesis.

In some embodiments, information comprises k-mer weights.

In some embodiments, the processor is further configured to: (i) perform with a computer system a sequence comparison between a sequencing read of the plurality of sequencing reads and a plurality of reference polynucleotide sequences, wherein the comparison comprises calculating k-mer weights as a measure of how likely it is that k-mers within the sequencing read are derived from a reference sequence within the plurality of reference polynucleotide sequences; (ii) identify the sequencing read as corresponding to a particular reference sequence in a database of reference sequences if the sum of k-mer weights for the reference sequence is above a threshold level; and (iii) assemble a record database comprising reference sequences identified in (ii), wherein the record database excludes reference sequences to which no sequencing read corresponds.

In some embodiments, the processor is further configured to: (i) for each sequencing read of the plurality of sequencing reads: (a) perform with a computer system a sequence comparison between a sequencing read and a plurality of reference polynucleotide sequences, wherein the comparison comprises calculating k-mer weights as a measure of how likely it is that k-mers within the sequencing read are derived from a reference sequence within the plurality of reference polynucleotide sequences; and (b) calculate a probability that the sequencing read corresponds to a particular reference sequence in a database of reference sequences based on the k-mer weights, thereby generating a sequence probability; (ii) calculate a score for the presence or absence of one or more taxa based on the sequence probabilities corresponding to sequences representative of the one or more taxa; and (iii) identify the one or more taxa as present or absent in the sample based on the corresponding scores.

In some embodiments, the entity indicator comprises a visual indicator, wherein the visual indicator displays sequencing read coverage. In some embodiments, a color, texture, pattern, uniqueness, or other demarcating feature is used to indicate a degree of sequencing read coverage. In some embodiments, the quality control indicator comprises a visual indicator, wherein the visual indicator displays the number of reads with a given read length or range of read lengths. In some embodiments, the visual indicator indicates a degree of uniqueness of a given sequence or k-mer.

In another aspect, the present disclosure provides a computer-implemented method for providing information corresponding to a sample, comprising: (a) providing data corresponding to the sample, wherein the data comprises a plurality of sequencing reads; (b) providing an interface to a user, wherein the interface displays to the user (i) an entity indicator indicating that the plurality of sequencing reads correspond to one or more entities, and (ii) a quality control indicator indicating the certainty with which the plurality of sequencing reads correspond to the one or more entities.

In some embodiments, the plurality of sequencing reads comprises deoxyribonucleic acid (DNA) sequencing reads and/or ribonucleic acid (RNA) sequencing reads. In some embodiments, the plurality of sequencing reads comprises both DNA sequencing reads and RNA sequencing reads. In some embodiments, the plurality of sequencing reads is generated using sequencing by synthesis, sequencing by ligation, nanopore sequencing, or sequencing by hybridization. In some embodiments, the plurality of sequencing reads is generated using sequencing by synthesis.

In some embodiments, an entity of the one or more entities is a human. In some embodiments, an entity of the one or more entities is a pathogen. In some embodiments, an entity of the one or more entities is selected from the group consisting of a fungus, bacterium, parasite, and virus. In some embodiments, one or more entities comprise a first entity that is a human and a second entity selected from the group consisting of a fungus, bacterium, parasite, and virus. In some embodiments, the second entity is associated with a disease or disorder. In some embodiments, the second entity is associated with an infection. In some embodiments, one or more additional entities are associated with a disease, disorder, or infection. In some embodiments, the one or more additional entities are selected from the group consisting of fungi, bacteria, parasites, and viruses. In some embodiments, the human has or is suspected of having a disease or disorder. In some embodiments, the human has been exposed or is suspected of having been exposed to a pathogen.

In some embodiments, the entity indicator comprises a visual indicator, wherein the visual indicator displays sequencing read coverage. In some embodiments, a color, texture, pattern, uniqueness, or other demarcating feature is used to indicate a degree of sequencing read coverage.

In some embodiments, the quality control indicator comprises a visual indicator, wherein the visual indicator displays the number of reads with a given read length or range of read lengths. In some embodiments, the visual indicator indicates a degree of uniqueness of a given sequence or k-mer.

In some embodiments, the method further comprises: (i) performing with a computer system a sequence comparison between a sequencing read of the plurality of sequencing reads and a plurality of reference polynucleotide sequences, wherein the comparison comprises calculating k-mer weights as a measure of how likely it is that k-mers within the sequencing read are derived from a reference sequence within the plurality of reference polynucleotide sequences; (ii) identifying the sequencing read as corresponding to a particular reference sequence in a database of reference sequences if the sum of k-mer weights for the reference sequence is above a threshold level; and (iii) assembling a record database comprising reference sequences identified in (ii), wherein the record database excludes reference sequences to which no sequencing read corresponds.

In some embodiments, the method further comprises: (i) for each sequencing read of the plurality of sequencing reads: (I) performing with a computer system a sequence comparison between a sequencing read and a plurality of reference polynucleotide sequences, wherein the comparison comprises calculating k-mer weights as a measure of how likely it is that k-mers within the sequencing read are derived from a reference sequence within the plurality of reference polynucleotide sequences; and (II) calculating a probability that the sequencing read corresponds to a particular reference sequence in a database of reference sequences based on the k-mer weights, thereby generating a sequence probability; (ii) calculating a score for the presence or absence of one or more taxa based on the sequence probabilities corresponding to sequences representative of the one or more taxa; and (iii) identifying the one or more taxa as present or absent in the sample based on the corresponding scores.

In another aspect, the present disclosure provides a system for providing information corresponding to a sample, comprising a processor configured to display the information on a web-based graphical interface, wherein the information is represented by one or more visual and/or textual indicators, including (i) an entity indicator, and (ii) a property indicator, wherein the information comprises one or more identities of one or more entities associated with the sample, wherein the entity indicator provides information about the one or more identities of the one or more entities, wherein the property indicator provides information about the properties of the one or more entities.

In some embodiments, a property of the one or more entities comprises an organism name. In some embodiments, a property of the one or more entities comprises a pathogen name. In some embodiments, a property of the one or more entities comprises a class type. In some embodiments, a property of the one or more entities comprises an RNA sensitive cutoff value. In some embodiments, a property of the one or more entities comprises an RNA specific cutoff value. In some embodiments, a property of the one or more entities comprises a DNA sensitive cutoff value. In some embodiments, a property of the one or more entities comprises a DNA specific cutoff value. In some embodiments, a property of the one or more entities comprises a validation indicator. In some embodiments, a property of the one or more entities comprises a medically relevant indicator. In some embodiments, a property of the one or more entities comprises one or more of publications associated with the one or more entities.

In some embodiments, the system further comprises a filter to reduce the number of the property indicators. In some embodiments, the filter is configured to filter using an average nucleotide identity value. In some embodiments, the filter is configured to filter using a percent coverage value. In some embodiments, the filter is configured to filter using read value. In some embodiments, the filter is configured to filter using a reference length value.

In some embodiments, the system further comprising a sample-level quality control indicator. In some embodiments, the sample-level quality indicator provides information about the one or more identities of the one or more entities. In some embodiments, the information comprises a total run yield value. In some embodiments, the information comprises a percentage of bases greater than or equal to Q30. In some embodiments, the information comprises a cluster density value.

In some embodiments, the system further comprises a run-level quality control indicator. In some embodiments, the run-level quality indicator provides information about the one or more identities of the one or more entities. In some embodiments, the information comprises a total raw read value. In some embodiments, the information comprises a unique read value. In some embodiments, the information comprises a post-adaptor reads value.

In some embodiments, an entity of the one or more entities is a human. In some embodiments, an entity of the one or more entities is a pathogen. In some embodiments, an entity of the one or more entities is selected from the group consisting of a fungus, bacterium, parasite, and virus. In some embodiments, the one or more entities comprise a first entity that is a human and a second entity selected from the group consisting of a fungus, bacterium, parasite, and virus. In some embodiments, the second entity is associated with a disease or disorder. In some embodiments, the second entity is associated with an infection. In some embodiments, a property of the one or more entities comprises an organism group. In some embodiments, the organism group is sorted.

In another aspect, the present disclosure provides a computer-implemented method for providing information corresponding to a sample. In some embodiments, the method comprises providing data corresponding to the sample, wherein the data comprises a plurality of sequencing reads. In some embodiments, the method comprises providing an interface to a user, wherein the interface displays to the user (i) an entity indicator indicating that the plurality of sequencing reads corresponds to one or more entities, and (ii) a property indicator indicating information about the properties of the one or more entities.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows an exemplary interface for an application.

FIGS. 2A and 2B show exemplary visualizations for sequencing quality control (QC) and processing control metrics, respectively.

FIG. 3 shows an exemplary visualization for sample quality control.

FIG. 4 shows an exemplary visualization for a quality control metric based on read length.

FIG. 5 shows an exemplary visualization for organism identification.

FIGS. 6A-6C show exemplary visualizations for coverage at various nucleotide positions at the gene level and at the genome level.

FIGS. 7A-7C show exemplary visualizations for quality control failure (FIG. 7A), organisms below cutoff in the positive processing control (FIG. 7B), and additional metrics for review (FIG. 7C).

FIGS. 8A and 8B show electrophoresis traces for quality control relating to adapter dimers.

FIGS. 9A and 9B show exemplary visualizations corresponding to repeat runs.

FIG. 10 shows an exemplary visualization for quality control metrics over many sequencing runs.

FIGS. 11A-11D show exemplary visualizations including filters for selecting species of interest (FIG. 11A), a frequency chart for organisms (FIG. 11B), a bar chart for organism types (FIG. 11C), and a bar chart showing changes in organisms over time (FIG. 11D).

FIG. 12 shows a computer system that is programmed or otherwise configured to implement methods of the present disclosure herein.

FIG. 13A-13D shows an exemplary visualization for the diagnostic test profile.

FIG. 14 shows an exemplary visualization for switching diagnostic test profile.

FIG. 15 shows an exemplary visualization that may allow a user to select a disease category using a graphical user interface.

FIG. 16 shows the number of publications on the web-based application user interface.

FIG. 17 shows an example of a list of publications from an external database.

FIG. 18 shows an exemplary visualization of a filter interface.

FIG. 19 shows an exemplary visualization of classifying organisms as members of a phylogenetically or semantically related group with the most likely organism shown at the top of the group tree view.

FIG. 20 shows an exemplary visualization of quality control metrics.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

Whenever the term “at most about” or “at least about” precedes the first numerical value in a series of two or more numerical values, the term “at most about” or “at least about” applies to each of the numerical values in that series of numerical values. For example, at most about 3, 2, or 1 is equivalent to at most about 3, at most about 2, or at most about 1.

The present disclosure provides systems and methods for providing information corresponding to a sample. A system for providing information corresponding to a sample may comprise a processor configured to display the information on a web-based graphical interface, wherein the information is represented by one or more visual and/or textual indicators (such as one or more graphs, bar charts, pie charts, scatter plots, 3D visualizations, text boxes, tables, or other indicators), including (i) an entity indicator, and (ii) a quality control indicator, wherein the information comprises the identities of one or more entities associated with the sample, wherein the entity indicator provides information about the identities of the one or more entities, and wherein the quality control indicator provides information about the certainty with which the identities of the one or more entities are determined. A method (e.g., a computer-implemented method) for providing information corresponding to a sample may comprise (a) providing data corresponding to the sample, wherein the data comprises a plurality of sequencing reads; (b) providing an interface to a user, wherein the interface displays to the user (i) an entity indicator (e.g., a visual and/or textual indicator) indicating that the plurality of sequencing reads correspond to one or more entities, and (ii) a quality control indicator (e.g., a visual and/or textual indicator) indicating the certainty with which the plurality of sequencing reads correspond to the one or more entities.

Entities corresponding to a sample may be, for example, a human and/or a microorganism. For example, an entity may be a human. In some cases, an entity may be a pathogen. An entity may be selected from the group consisting of a fungus, bacterium, parasite, and virus. In some cases, the one or more entities associated with a sample may comprise a first entity that is a human and a second entity selected from the group consisting of a fungus, bacterium, parasite, and virus. The second entity, and/or one or more other entities, may be associated with a disease or disorder, such as an infection. For example, the second entity may be associated with a disease or disorder, and/or the second entity and a third entity (e.g., another fungus, bacterium, parasite, or virus) may be associated with a disease or disorder. A sample may derive from a patient (e.g., a human patient). A patient from which a sample derives may have or be suspected of having a disease or disorder. In some cases, a patient from which a sample derives may have or be suspected of having a disease or disorder associated with a pathogen (e.g., bacteria, fungi, parasite, or virus). In some cases, a patient from which a sample derives may have been exposed or be suspected of having been exposed to a pathogen.

A sample may comprise a bodily fluid, such as blood, urine, saliva, or sweat. A sample may comprise one or more cells, and/or may comprise cell-free nucleic acid molecules. Cells of a sample may be lysed to provide access to a plurality of nucleic acid molecules therein.

A plurality of sequencing reads may be derived from a sample. The plurality of sequencing reads may correspond to the one or more entities associated with the sample. The plurality of sequencing reads may comprise deoxyribonucleic acid (DNA) sequencing reads and/or ribonucleic acid (RNA) sequencing reads. In some cases, the plurality of sequencing reads may comprise both DNA sequencing reads and RNA sequencing reads. The plurality of sequencing reads may be generated from nucleic acid molecules included within the sample using, for example, sequencing by synthesis, sequencing by ligation, nanopore sequencing, or sequencing by hybridization.

### K-mer Based Analysis

Information corresponding to a sample may comprise or be derived from k-mer weights. In general, a sequencing read (also referred to as a “read” or “query sequence”) refers to the inferred sequence of nucleotide bases in a nucleic acid molecule. A sequencing read may be of any appropriate length, such as about or more than about 20 nt, 30 nt, 36 nt, 40 nt, 50 nt, 75 nt, 100 nt, 150 nt, 200 nt, 250 nt, 300 nt, 400 nt, 500 nt, or more in length. In some embodiments, a sequencing read is less than 200 nt, 150 nt, 100 nt, 75 nt, or fewer in length. Sequencing reads can be “paired,” meaning that they are derived from different ends of a nucleic acid fragment. Paired reads can have intervening unknown sequence or overlap. In some cases, the sequencing read is a contig or consensus sequence assembled from separate overlapping reads. A sequencing read may be analyzed in terms of component k-mers. In general, “k-mer” refers to the subsequences of a given length k that make up a sequencing read. For example, a sequence “AGCTCT” can be divided into the 3-nt subsequences “AGC,” “GCT,” “CTC,” and “TCT.” In this example, each of these subsequences is a k-mer, wherein k=3. K-mers may be overlapping or non-overlapping.

Sequence comparison may comprise one or more comparison steps in which one or more k-mers of a sequencing read are compared to k-mers of one or more reference sequences (also referred to simply as a “reference”). In some embodiments, a k-mer is about or more than about 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, 30 nt, 35 nt, 40 nt, 45 nt, 50 nt, 75 nt, 100 nt, or more in length. In some embodiments, a k-mer is about or less than about 30 nt, 25 nt, 20 nt, 15 nt, 10 nt, or fewer in length. The k-mer may be in the range of 3 nt to 13 nt, 5 nt to 25 nt in length, 7 nt to 99 nt, or 3 nt to 99 nt in length. The length of k-mer analyzed at each step may vary. For example, a first comparison may compare k-mers in a sequencing read and a reference sequence that are 21 nt in length, whereas a second comparison may compare k-mers in a sequencing read and a reference sequence that are 7 nt in length. For any given sequence in a comparison step, k-mers analyzed may be overlapping (such as in a sliding window), and may be of same or different lengths. While k-mers are generally referred to herein as nucleic acid sequences, sequence comparison also encompasses comparison of polypeptide sequences, including comparison of k-mers consisting of amino acids.

In some cases, a processor of a system for providing information corresponding to a sample may be configured to: (i) perform with a computer system a sequence comparison between a sequencing read of the plurality of sequencing reads and a plurality of reference polynucleotide sequences, wherein the comparison comprises calculating k-mer weights as a measure of how likely it is that k-mers within the sequencing read are derived from a reference sequence within the plurality of reference polynucleotide sequences; (ii) identify the sequencing read as corresponding to a particular reference sequence in a database of reference sequences if the sum of k-mer weights for the reference sequence is above a threshold level; and (iii) assemble a record database comprising reference sequences identified in (ii), wherein the record database excludes reference sequences to which no sequencing read corresponds. Alternatively or in addition, the processor may be configured to: (i) for each sequencing read of the plurality of sequencing reads: (a) perform with a computer system a sequence comparison between a sequencing read and a plurality of reference polynucleotide sequences, wherein the comparison comprises calculating k-mer weights as a measure of how likely it is that k-mers within the sequencing read are derived from a reference sequence within the plurality of reference polynucleotide sequences; and (b) calculate a probability that the sequencing read corresponds to a particular reference sequence in a database of reference sequences based on the k-mer weights, thereby generating a sequence probability; (ii) calculate a score for the presence or absence of one or more taxa based on the sequence probabilities corresponding to sequences representative of the one or more taxa; and (iii) identify the one or more taxa as present or absent in the sample based on the corresponding scores.

In some cases, a computer-implemented method for providing information corresponding to a sample may comprise: (i) performing with a computer system a sequence comparison between a sequencing read of the plurality of sequencing reads and a plurality of reference polynucleotide sequences, wherein the comparison comprises calculating k-mer weights as a measure of how likely it is that k-mers within the sequencing read are derived from a reference sequence within the plurality of reference polynucleotide sequences; (ii) identifying the sequencing read as corresponding to a particular reference sequence in a database of reference sequences if the sum of k-mer weights for the reference sequence is above a threshold level; and (iii) assembling a record database comprising reference sequences identified in (ii), wherein the record database excludes reference sequences to which no sequencing read corresponds. Alternatively or in addition, the method may comprise: (i) for each sequencing read of the plurality of sequencing reads: (I) performing with a computer system a sequence comparison between a sequencing read and a plurality of reference polynucleotide sequences, wherein the comparison comprises calculating k-mer weights as a measure of how likely it is that k-mers within the sequencing read are derived from a reference sequence within the plurality of reference polynucleotide sequences; and (II) calculating a probability that the sequencing read corresponds to a particular reference sequence in a database of reference sequences based on the k-mer weights, thereby generating a sequence probability; (ii) calculating a score for the presence or absence of one or more taxa based on the sequence probabilities corresponding to sequences representative of the one or more taxa; and (iii) identifying the one or more taxa as present or absent in the sample based on the corresponding scores.

A reference sequence may include any sequence to which a sequencing read is compared. Typically, the reference sequence is associated with some known characteristic, such as a condition of a sample source, a taxonomic group, a particular species, an expression profile, a particular gene, an associated phenotype such as likely disease progression, drug resistance or pathogenicity, increased or reduced predisposition to disease, or other characteristic. Typically, a reference sequence is one of many such reference sequences in a database. A variety of databases comprising various types of reference sequences are available, one or more of which may serve as a reference database either individually or in various combinations. Databases can comprise many species and sequence types, such as NR, UniProt, SwissProt, TrEMBL, or UniRef90. Databases can comprise specific kinds of sequences from multiple species, such as those used for taxonomic classification of species, such as bacteria. Such databases can be 16S databases, such as the Greengenes database, the UNITE database, or the SILVA database. Marker genes other than 16S ribosomal RNA (rRNA) may be used as reference sequences for the identification of microorganisms (e.g. bacteria), such as metabolic genes, genes encoding structural proteins, proteins that control growth, cell cycle or reproductive regulation, housekeeping genes or genes that encode virulence, toxins, or other pathogenic factors. Specific examples of marker genes other than 16S rRNA include, but are not limited to, 18S ribosomal DNA (rDNA), 23S rDNA, gyrA, gyrB gene, groEL, rpoB gene, fusA gene, recA gene, sod A, cox1 gene, and nifD gene. Reference databases can comprise internal transcribed sequences (ITS) databases, such as UNITE, ITSoneDB, or ITS2. Databases can comprise multiple sequences from a single species, such as the human genome, the human transcriptome, model organisms such as the mouse genome, the yeast transcriptome, or the C. elegans proteome, or disease vectors such as bat, tick, or mosquitoes and other domestic and wild animals. In some embodiments, the reference database comprises sequences of human transcripts. Reference sequences in databases can comprise DNA sequences, RNA sequences, or protein sequences. Reference sequences in databases can comprise sequences from a plurality of taxa. In some cases, the reference sequences are from a reference individual or a reference sample source. Examples of reference individual genomes are, for example, a maternal genome, a paternal genome, or the genome of a non-cancerous tissue sample. Examples of reference individuals or sample sources are the human genome, the mouse genome, or the genomes of particular serovars, genovars, strains, variants or otherwise characterized types of bacteria, archea, viruses, phages, fungi, and parasites. The database can comprise polymorphic reference sequences that contain one or more mutations with respect to known polynucleotide sequences. Such polymorphic reference sequences can be different alleles found in the population, such as SNPs, indels, microdeletions, microexpansions, common rearrangements, genetic recombinations, or prophage insertion sites, and may contain information on their relative abundance compared to non-polymorphic sequences. Polymorphic reference sequences may also be artificially generated from the reference sequences of a database, such as by varying one or more (including all) positions in a reference genome such that a plurality of possible mutations not in the actual reference database are represented for comparison. The database of reference sequences can comprise reference sequences of one or more of a variety of different taxonomic groups, including but not limited to bacteria, archaea, chromalveolata, viruses, fungi, plants, fish, amphibians, reptiles, birds, mammals, and humans. In some cases, the database of reference sequences consists of sequences from one or more reference individuals or a reference sample sources (e.g. 10, 100, 1000, 10000, 100000, 1000000, or more), and each reference sequence in the database is associated with its corresponding individual or sample source. In some embodiments, an unknown sample may be identified as originating from an individual or sample source represented in the reference database on the basis of a sequence comparison.

In some embodiments, each reference sequence in the database of reference sequences is associated with, prior to the comparison, a k-mer weight as a measure of how likely it is that a k-mer within the reference sequence originates from the reference sequence. Alternatively, the database of reference sequences can comprise sequences from a plurality of taxa, and each reference sequence in the database of reference sequences is associated with a k-mer weight as a measure of how likely it is that a k-mer within the reference sequence originates from a taxon within the plurality of taxa. Calculating the k-mer weight can comprise comparing a reference sequence in the database to the other reference sequences in the database, such as by a method described herein. The k-mer values thus associated with sequences or taxa in the database may then be used in determining k-mer weights for k-mers within sequencing reads.

In general, comparing k-mers in a read to a reference sequence comprises counting k-mer matches between the two. The stringency for identifying a match may vary. For example, a match may be an exact match, in which the nucleotide sequence of the k-mer from the read is identical to the nucleotide sequence of the k-mer from the reference. Alternatively, a match may be an incomplete match, where 1, 2, 3, 4, 5, 10, or more mismatches are permitted. In addition to counting matches, a likelihood (also referred to as a “k-mer weight” or “KW”) can be calculated. In some embodiments, the k-mer weight relates a count of a particular k-mer within a particular reference sequence, a count of the particular k-mer among a group of sequences comprising the reference sequence, and a count of the particular k-mer among all reference sequences in the database of reference sequences. In one embodiment, the k-mer weight is calculated according to the following formula, which calculates the k-mer weight as a measure of how likely it is that a particular k-mer (Kᵢ) originates from a reference sequence (refᵢ) as follows:

$${KEref}_{i} ( {K}_{i} ) = \frac{{C}_{ref} ( {K}_{i} ) / {C}_{db} ( {K}_{i} )}{{C}_{db} ( {K}_{i} ) / Total kmer count} \qquad ( Eqn . 1 )$$

C represents a function that returns the count of Kᵢ. C_{ref}(Kᵢ) indicates the count of the Kᵢ in a particular reference. C_{db}(Kᵢ) indicates the count of Kᵢ in the database. This weight provides a relative, database specific measure of how likely it is that a k-mer originated from a particular reference. Prior to comparing a sequencing read to the database of reference sequences, the k-mer weight (or measurement of likelihood that a k-mer originates from a given reference sequence) can be calculated for each k-mer and reference sequence in the database. In some cases, when a reference databases comprises sequences from a plurality of taxa, each reference sequence can be associated with a measure of likelihood, or k-mer weight, that a k-mer within the reference sequence originates from a taxon within a plurality of taxa. As a non-limiting example, a reference database can comprise sequences from multiple species of canines, and the k-mer weight could be calculated by relating the count of a given k-mer in all canine sequences to its count in the entire database, which includes other taxa. In some examples, the k-mer weight measuring how likely it is that a k-mer originates from a specific taxon is calculated by defining C_{ref}(Kᵢ) in the above equation as a function that returns the total count of Kᵢ in a particular taxon.

For each reference sequence, reference database derived weights for a plurality of k-mers within a sequencing read may be added and compared to a threshold value. The threshold value can be specific to the collection of reference sequences in the database and may be selected based on a variety of factors, such as average read length, whether a specific sequence or source organism is to be identified as present in the sample, and the like. If the sum of k-mer weights for the reference sequence is above the threshold level, the sequencing read may be identified as corresponding to the reference sequence, and optionally the organism or taxonomic group associated with the reference sequence. In some cases, the read is assigned to the reference sequence with the maximum sum of k-mer weights, which may or may not be required to be above a threshold. In the case of a tie, where a sequence read has an equal likelihood of belonging to more than one reference sequence as measured by k-mer weight, the sequence read can be assigned to the taxonomic lowest common ancestor (LCA) taking into account the read's total k-mer weight along each branch of the phylogenetic tree. In general, correspondence of a sequence read with a reference sequence, organism, or taxonomic group indicates that the reference sequence, organism, or taxonomic group was present in the sample.

In some aspects, the methods comprise calculating a probability. In some cases, a probability is calculated for a sequencing read generated from a plurality of polynucleotides. In some cases, the probability is the probability (or likelihood) that the sequencing read corresponds to a particular reference sequence in a database of reference sequences based on the k-mer weights. A probability may be calculated for each sequencing read, thereby generating a plurality of sequence probabilities. In some cases, the presence or absence of one or more taxa in a sample may be determined based on the sequence probabilities. For example, the probability may identify a first bacterial strain as being present in the sample and a second bacterial strain as being absent in the sample. In some cases, the probability is represented as a percentage (%) or as a fraction. In some cases, a probability is provided as a score representative of the probability. The score can be based on any arbitrary scale so long as the score is indicative of the probability (e.g. a probability that an individual sequence corresponds to a particular reference sequence, or a probability that a particular taxon is present in the sample). The probability or a score representative of the probability may be used to determine the presence or absence of one or more taxa within a sample. For example, a probability or score above a threshold value may be indicative of presence, and/or a probability or score below a threshold value may be indicative of absence. In some embodiments, presence or absence is reported as a probability, rather than an absolute call. Example methods for calculating such probabilities are provided herein. In general, embodiments described herein in terms of presence or absence likewise encompass calculating a probability or score for such presence or absence.

Results of methods described herein will typically be assembled in a record database. In some embodiments, the record database comprises reference sequences identified as present in the sample and excludes reference sequences to which no sequencing read was found to correspond, such as by failure to match a sequencing read above a set threshold level. The software routines used to generate the sequence record database and to compare sequencing reads to the database can be run on a computer. The comparison can be performed automatically upon receiving data. The comparison can be performed in response to a user request. The user request can specify which reference database to compare the sample to. The computer can comprise one or more processors. Processors may be associated with one or more controllers, calculation units, and/or other units of a computer system, or implanted in firmware as desired. If implemented in software, the routines may be stored in any computer readable memory, such as in RAM, ROM, flash memory, a magnetic disk, a laser disk, or other storage medium. The record database, sequencing reads, or a report summarizing the results of database construction or sequence read comparison may also be stored in any suitable medium, such as in RAM, ROM, flash memory, a magnetic disk, a laser disk, or other storage medium. Likewise, the record database, sequencing reads, or a report summarizing the results of database construction or sequence read comparison may be delivered to a computing device via any known delivery method including, for example, over a communication channel such as a telephone line, the internet, a wireless connection, etc., or via a transportable medium, such as a computer readable disk, flash drive, etc. . . . . A database, sequencing reads, or report may be communicated to a user at a local or remote location using any suitable communication medium. For example, the communication medium can be a network connection, a wireless connection, or an internet connection. A database or report can be transmitted over such networks or connections (or any other suitable means for transmitting information, including but not limited to mailing database summary, such as a print-out) for reception and/or for review by a user. The recipient can be but is not limited to the customer, an individual, a health care provider, a health care manager, or electronic system (e.g. one or more computers, and/or one or more servers). In some embodiments, the database or report generator sends the report to a recipient's device, such as a personal computer, phone, tablet, or other device. The database or report may be viewed online, saved on the recipient's device, or printed. The comparison of communicated sequencing reads to a database can occur after all the reads are uploaded. The comparison of communicated sequencing reads to a database can begin while the sequencing reads are in the process of being uploaded.

One or more steps of a method described herein may be performed in parallel for each of the plurality of sequencing reads. For example, each of the sequencing reads in the plurality may be subjected in parallel to a first sequence comparison between the sequencing read and a plurality of reference polynucleotide sequences (e.g. reference polynucleotide sequences from a plurality of different taxa and/or a plurality of different reference databases). Comparison in parallel differs from certain stepwise comparison processes in that sequencing reads having a purported match in a first reference database are not subtracted from the query set of sequences for subsequent comparison with a second reference database. In such a stepwise process, sequences having a purported match in the first database may be incorrectly identified before comparison being run against a reference database containing a more accurate match (e.g. the correct sequence). Instead, by running a comparison against a plurality of different reference sequences corresponding to a plurality of different taxa, each sequence can be assigned to an optimal first taxonomic class prior to identifying with greater specificity a sequence or taxon to which a sequencing read corresponds. For example, sequencing reads may be first classified as corresponding to human, bacterial, or fungal sequences before identifying a particular gene, bacterial species, or fungal species to which the sequencing read corresponds. In some instances, this process is referred to as “binning.” Parallel sequence comparison may comprise comparison with sequences from two or more different taxonomic groups, such as 3, 4, 5, 6, or more different taxonomic groups. In some embodiments, the different taxonomic groups may be selected from two or more of the following bacteria, archaea, chromalveolata, viruses, fungi, plants, fish, amphibians, reptiles, birds, mammals, and humans.

In some embodiments, a method may further comprise quantifying an amount of polynucleotides corresponding to a reference sequence identified in an earlier step. Quantification can be based on a number of corresponding sequencing reads identified. This can include normalizing the count by the total number of reads, the total number of reads associated with sequences, the length of the reference sequence, or a combination thereof. Examples of such normalization include FPKM and RPKM, but may also include other methods that take into account the relative amount of reads in different samples, such as normalizing sequencing reads from samples by the median of ratios of observed counts per sequence. A difference in quantity between samples can indicate a difference between the two samples. The quantitation can be used to identify differences between subjects, such as comparing the taxa present in the microbiota of subjects with different diets, or to observe changes in the same subject over time, such as observing the taxa present in the microbiota of a subject before and after going on a particular diet.

The presence, absence, or abundance of particular sequences, polymorphisms, or taxa can be used for diagnostic purposes, such as inferring that a sample or subject associated with the sample has a particular condition (e.g. an illness), has had a particular condition, or is likely to develop a particular condition if sequence reads associated with the condition (e.g. from a particular disease-causing organism) are present at higher levels than a control (e.g. an uninfected individual). In another embodiment, the sequencing reads can originate from the host and indicate the presence of a disease-causing organism by measuring the presence, absence, or abundance of a host gene in a sample. The presence, absence, or abundance can be used to determine the need for a treatment or care intensity, inform the choice of a treatment, infer effectiveness of a treatment, wherein a decrease in the number of sequencing reads from a disease-causing agent after treatment, or a change in the presence, absence, or abundance of specific host-response genes, indicates that a treatment is effective, whereas no change or insufficient change indicates that the treatment is ineffective. The sample can be assayed before or one or more times after treatment is begun. In some examples, the treatment of the infected subject is altered based on the results of the monitoring.

In some cases, one or more samples (e.g. blood, plasma, other body fluids, tissues, swab samples etc.) having a known condition may be used to establish a biosignature for that condition. The biosignature may be established by associating the record database with the condition. The condition can be any condition described herein. For example, a plurality of samples from a particular environmental source may be used to identify sequences and/or taxa associated with that environmental source, thereby establishing a biosignature consisting of those sequences and/or taxa so associated. In general, the term “biosignature” is used to refer to an association of the presence, absence, or abundance of a plurality of sequences and/or taxa with a particular condition, such as a classification, diagnosis, prognosis, and/or predicted outcome of a condition in a subject; a sample source; contamination by one or more contaminants; or other condition. A biosignature may be used as a reference database associated with a condition for the identification of that condition in another sample. In one embodiment, the establishing the biosignature comprises a determination of the presence, absence, and/or quantity of at least 10, 50, 100, 1000, 10000, 100000, 1000000, or more sequences and/or taxa in a sample using a single assay. Establishing a biosignature may comprise comparing sequencing reads for one or more samples representative of the condition with one or more samples not representative of the condition. For example, a biosignature can consist of gene expression involved in a host response (e.g. an immune response) among individuals infected by a virus, which sequences may be compared to sequences from subjects that are not infected or are infected by some other agent (e.g. bacteria). In such case, the presence, absence, or abundance of particular sequencing reads may be associated with a viral rather than a bacterial infection. In another example, the biosignature can consist of sequences of genes involved in a variety of antiviral responses, the presence, absence, or abundance of sequencing reads associated with which can be indicative of a specific class or type of viral infection. In some embodiments, the biosignature associated with a reference database consists of the sequences (and optionally levels) of host transcripts and/or the sequences (and optionally levels) of transcripts or genomes of one or more infectious agents. In one particular example, the condition is influenza infection and the biosignature consists of sequences of one or more of (e.g. 1, 2, 3, 4, 5, 6, 7, 8, 9, 10, 11, 12, or all of) IFIT1, IFI6, IFIT2, ISG15, OASL, IFIT3, NT5C3A, MX2, IFITM1, CXCL10, IFI44L, MX1, IFIH1, OAS2, SAMD9, RSAD2, and DDX58. In another example, the reference database could be common mutations or gene fusions found in cancerous cells, and the presence, absence, or abundance of sequencing reads associated with the biosignature can indicate that the patient has or does not have detectable cancer, what type of cancer a detectable cancer is, a preferred treatment method, whether existing treatment is effective, and/or prognosis.

### Presentation of Sample Information

Information about a sample, such as information regarding entities associated with the sample, may be presented using a software program or platform. A software platform may comprise one or more components, such as a component for providing information about a sample, a component for analyzing sequencing information (e.g., performing a k-mer based analysis), a component for analyzing and classifying processed sequencing reads, and a component for supporting laboratory sample preparation. The software program is an exemplary platform that includes three such components: a review portal which is a web browser accessible dashboard application; an analysis pipeline which processes raw NGS data for analysis by the classification algorithm; and the sequence portal web-based application which supports sample information entry and laboratory sample preparation.

In some cases, information about a sample may be provided via a web-based interface. A web-based interface may be accessible using any web browser. A web-based interface may be accessible from a computing device, such as a personal or portable computing device or a stationary device. In some cases, a web-based interface may be accessible from a computer disposed in a laboratory, hospital, clinic, or other setting. Certain features of the web-based interface may be accessible without a network (e.g., internet) connection. For example, stored information about a previously analyzed sample may be accessible without a network connection. In some cases, information may be locally stored and accessible from the web-based interface with or without a network connection.

A web-based application may comprise one or more sections that may be accessible from a main page or portal. The application may comprise a menu (e.g., a drop down menu, tabular menu, list, menu bar, or other menu) facilitating navigation between multiple sections. The menu may be accessible from some or all pages or sections of the application. For example, the menu may be accessible from the same location of each page or section. The one or more sections of a web-based application may include a main page or portal (e.g., a home page) from which a user may select to navigate to another section. For example, the main page or portal may comprise a log-in feature where a user may provide an assigned username and password to obtain access to the application. A user may select to view a particular report, such as a report associated with a given patient and/or sample. Report selection may be made, for example, in a section of the application accessible from a main page or portal.

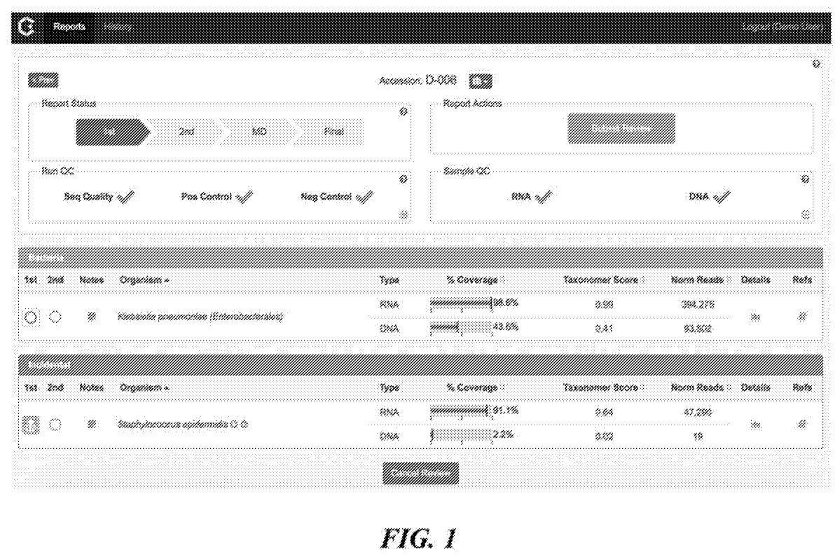

A dashboard software application accessible from a web browser may enable detailed review of pathogens detected by a novel infectious disease diagnostic test based on, for example, methods and systems described elsewhere herein, specifically Taxonomer organism classification. Test results unique to methods and systems described elsewhere herein may be displayed for each suspected pathogen in an individual patient, in concert with QC assessment of the underlying next-generation sequencing (NGS) data and controls. FIG. 1 displays an exemplary interface for such an application. As shown in FIG. 1, the interface may comprise details of a report status (e.g., an indication of how many levels of review it has undergone by one or more scientists, technicians, medical professionals, doctors, or other reviews), assessments performed (e.g., quality control assessments), and entity identities. The report may also indicate whether both RNA and DNA sequencing reads have been analyzed. Entity identities may be indicated graphically and/or textually. In some cases, an entity indicator may comprise a display corresponding to RNA analysis and a display corresponding to DNA analysis.

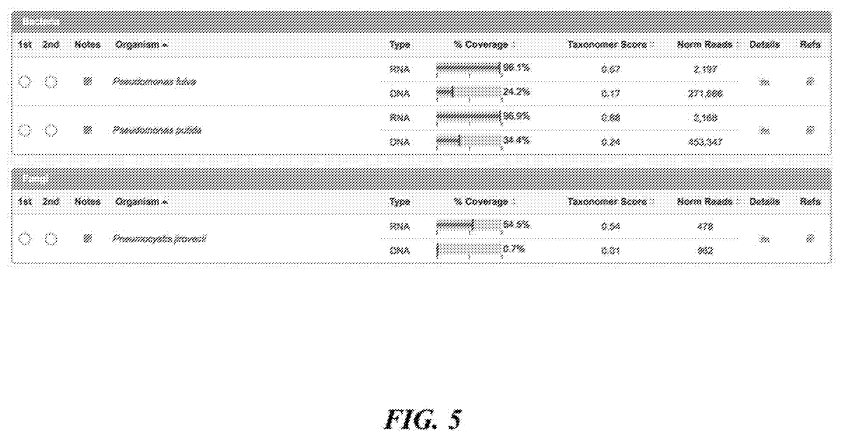

The methods and systems provided herein may facilitate identification of one or more entities (e.g., organisms) within a sample. FIG. 5 shows an exemplary visualization for organism identification. As shown in FIG. 5, organisms may be grouped categorically (e.g., bacteria, fungi, and viruses).

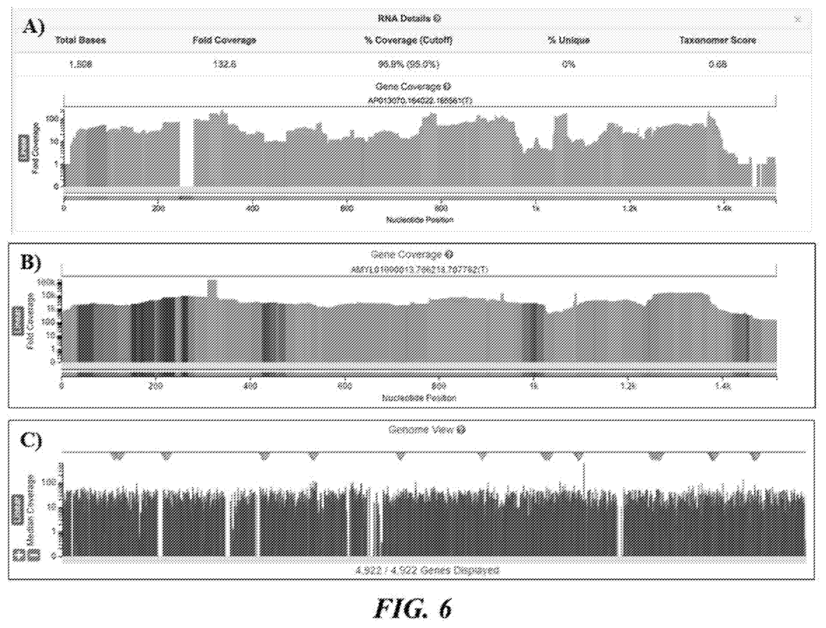

The results metrics of a diagnostic test, calculated from an organism classification algorithm, may be presented for each entity (such as each suspected pathogen) in a novel display, where sequencing read coverage is shown as bars along the genome or a gene, and the darker color of the bars represents the uniqueness of the regions of the reference genome or a gene. FIGS. 6A-6C show exemplary visualizations for coverage at various nucleotide positions at the gene and genome levels. Results may be displayed based on k-mer analysis of sequencing read coverage, rather than sequencing reads. The total number of bases in a reference sequence, average number of estimated reads at each position along the reference sequence (fold coverage), minimum coverage required to display organism detection (% coverage), percentage of sequences unique to an organism as detected by the analysis software (e.g., Taxonomer) (% unique), and/or a Taxonomer Score may also be provided. In some cases, a gene coverage plot such as that shown in FIG. 6B may display coverage depth at each base for the 16S/18S gene. A darker shade may signify a more unique portion of the gene, while gray areas may indicate less unique portions. The most unique portions may be highlighted by an additional indicator, such as a different color, texture, or pattern. The uniqueness indicated by such a gene coverage plot may be based on k-mer analysis (e.g., as described herein). In some cases, a genome view plot may be provided to allow visualization of an entire genome of an organism (FIG. 6C). The plot may display the median coverage depth for each gene. Genes with a higher total percent coverage may be indicated by, for example, a particular color, texture, or pattern.

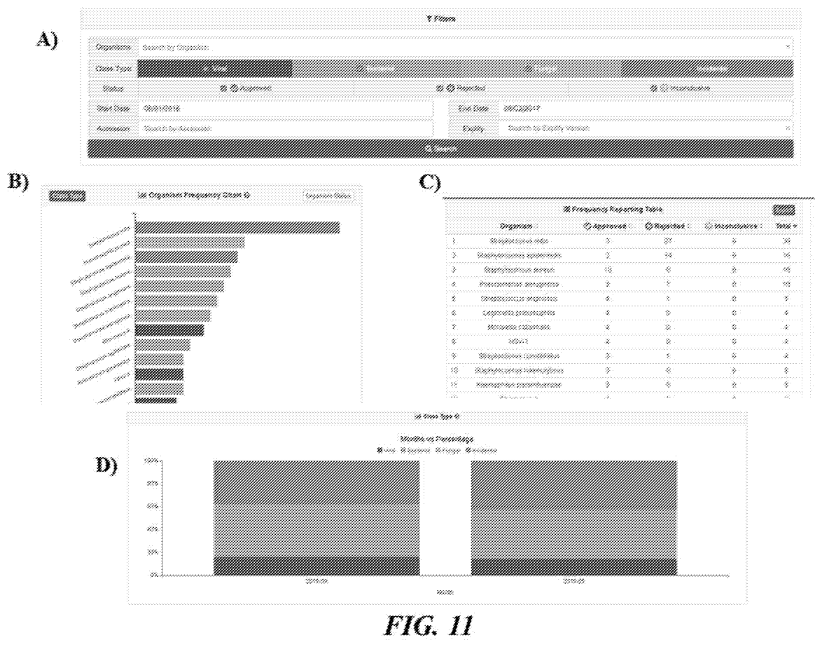

Results corresponding to sample information may be provided in a summary view. FIGS. 11A-11C show exemplary visualizations including filters for selecting species of interest (FIG. 11A), a frequency chart for organisms (FIG. 11B), and a bar chart for organism types (FIG. 11C). These metrics may be provided in a separate section of the web-based application.

The web-based application may also provide numerous quality control indicators for analyzing the quality of an analysis corresponding to a given sample. Different types of quality control indicators may be provided in different sections of the web-based application. Alternatively, all quality control indicators may be available in the same section of the application. In some cases, a user may choose to view or hide a given quality control metric, such as a visualization or other indicator. In some cases, the application may display pre-determined quality control metrics that may be selected by, for example, an administrator. In this case, quality control metrics may not be selectively filtered by any user but may only be changed by the administrator. The administrator may attain access to an editable version of the application by signing in to the application with an appropriate username and password.

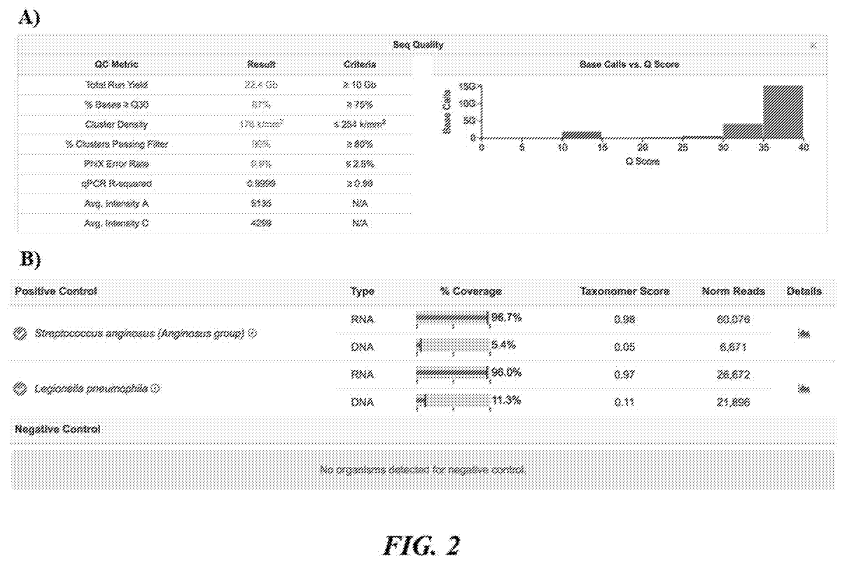

FIGS. 2A and 2B show exemplary visualizations for sequencing quality control and processing control metrics, respectively. Quality metrics may include, for example, total run yield, cluster density, and other metrics and may be displayed alongside threshold metrics. Sequencing quality may also be indicated using a visualization displaying base calls relative to Q score, as shown in FIG. 2A. As shown in FIG. 2B, external processing controls (e.g., one or more positive or negative controls) may also be used to assess sequencing quality. The diagnostic test may use processing control samples that are run in parallel with patient samples, and a set of control organisms that may be added to all samples at the start of the laboratory sample preparation. The results from these external processing controls and internal control organisms are presented in novel ways in the context of assessing QC, estimating the level of test sensitivity, and reviewing individual suspected pathogens.

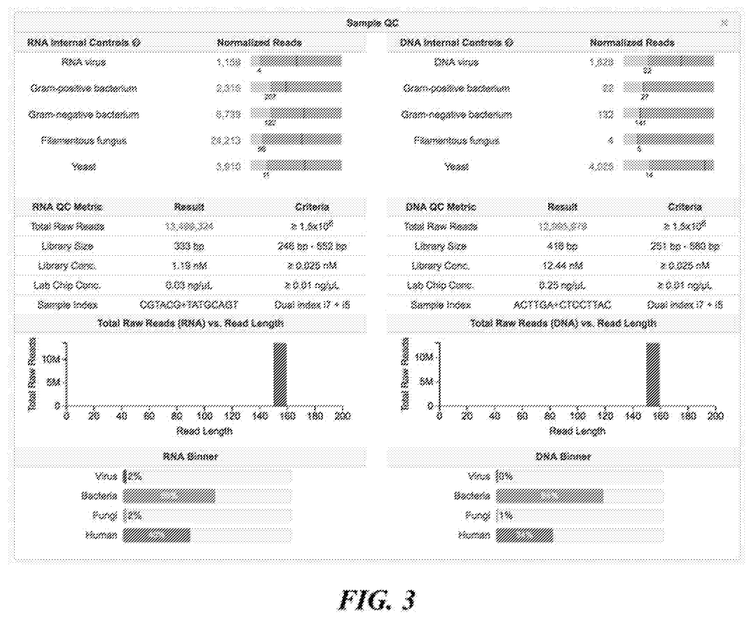

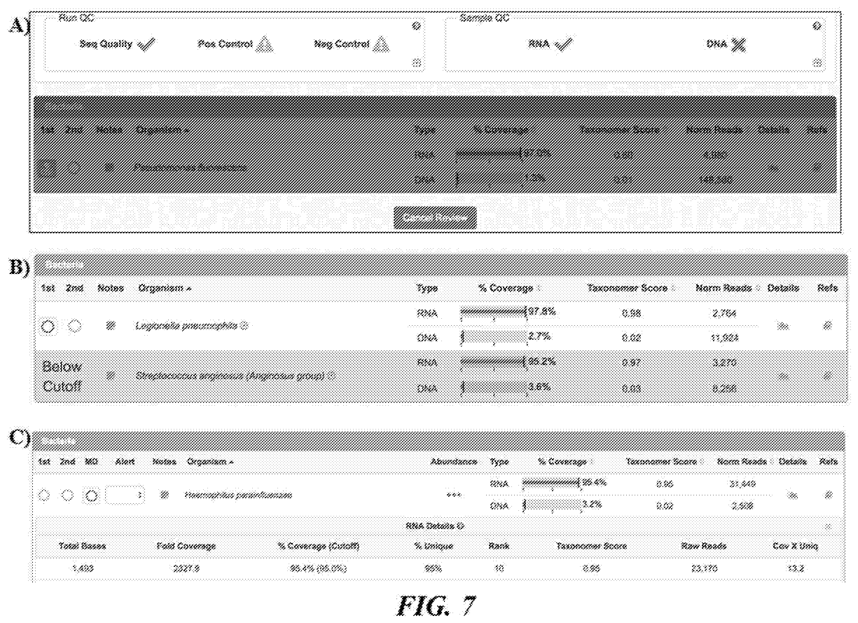

FIG. 3 shows another exemplary visualization for sample quality control. Sample quality control metrics may be tracked for a given analysis (e.g., run) of a given sample. Sample quality control may be assessed separately for RNA and DNA. One or more indicators may be used to indicate that controls pass or do not pass a quality control check. FIGS. 7A-7C show exemplary visualizations for quality control failure (FIG. 7A), organisms below cutoff in the positive processing control (FIG. 7B), and additional metrics for review (FIG. 7C).

The laboratory procedure creates sample libraries for sequencing; for the Illumina NGS platform, short double stranded adaptors are ligated to fragments of sample DNA. Combinations of adaptors containing different short index sequences may be randomly assigned to samples in a novel manner to mitigate contamination of data from previous sequencing runs. The application may provide a novel user interface to make manual changes to these assignments.

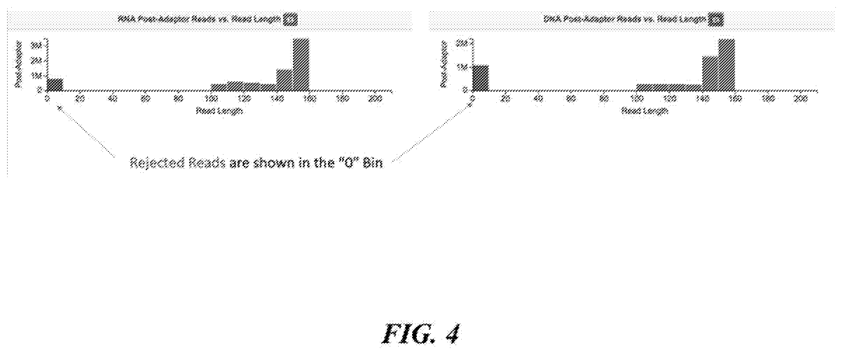

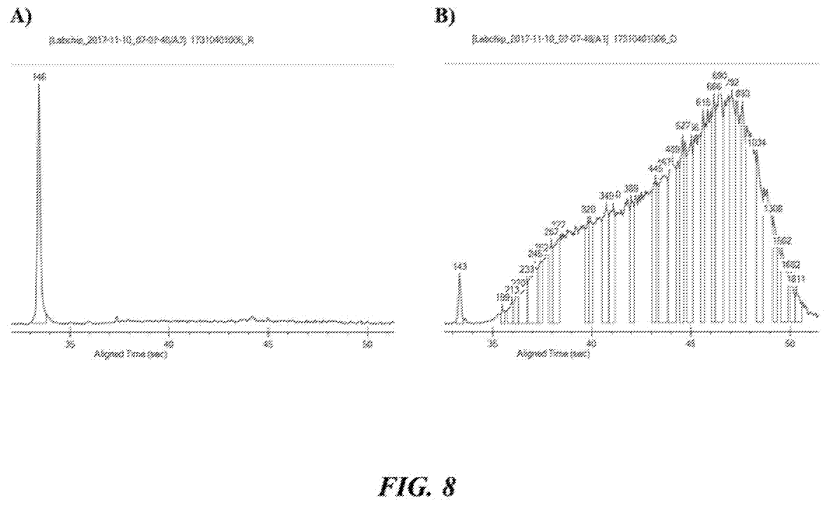

Adaptors can form non-informative dimers which are typically measured in the laboratory using electrophoresis methods. As part of quality control assessment, the occurrence of adaptor-dimers may be displayed in a novel view in the dashboard application and can serve as an in-silico alternative to electrophoresis (FIG. 4). Reads may be rejected if there are adapter sequences present. FIGS. 8A and 8B show electrophoresis traces for quality control relating to adapter dimers. In FIGS. 8A and 8B, the majority of rejected reads are due to adapter-dimers which appear in electrophoresis traces at around 145 base pairs.

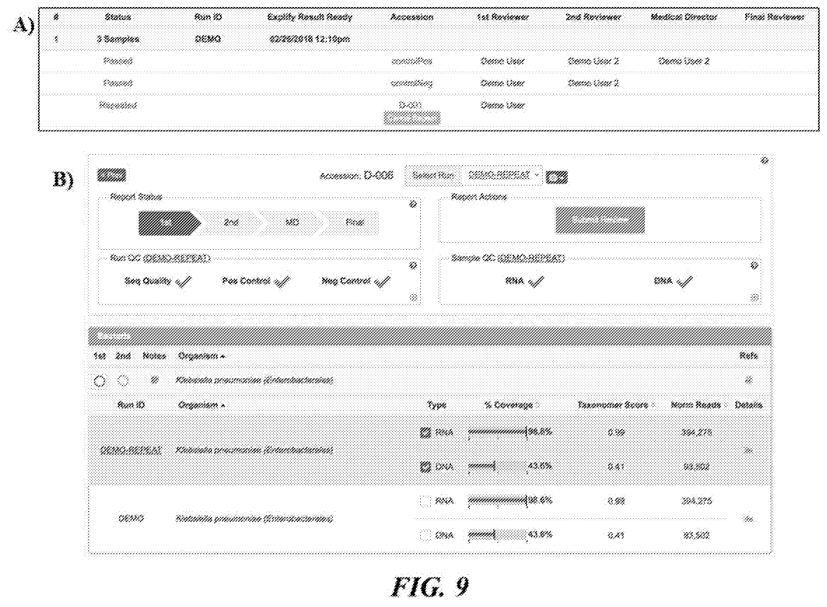

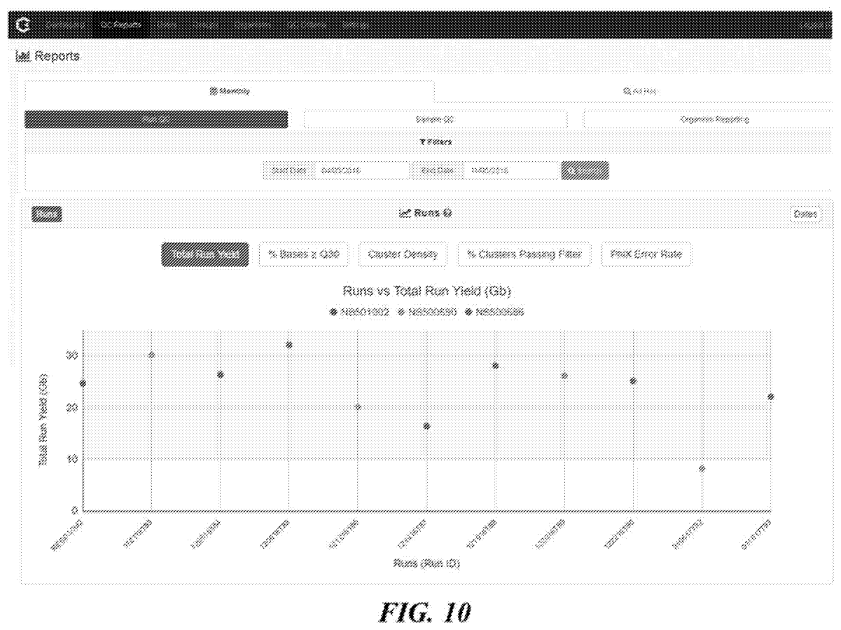

Occasionally a test may be repeated, resulting in more than one set of results for a given patient sample. The multiple sets of sequencing quality control data and analysis results may be presented in a novel way that allows a union view of the original set alongside newer sets from repeats. FIGS. 9A and 9B show exemplary visualizations corresponding to repeat runs, and FIG. 10 shows an exemplary visualization for quality control metrics relating to repeated sequencing runs.

The dashboard application may support a workflow for, for example, diagnostic decision making. The workflow may involve multiple reviewers having different roles, such as technologist and medical director, through the novel use of visual elements that guide the review process and enforce workflow policies. For example, a report corresponding to a sample (e.g., a sample associated with a given patient) may be accessed through the interface by a technologist. The technologist may review the report and determine whether they agree with the report and/or believe that the data is of sufficient quality. They may enter their conclusions, as well as notes regarding their determination (e.g., whether another run should be performed, whether they draw any particular medical conclusion from the results, etc.), into an interface of the application. The report may also be analyzed by one or more additional users, including a doctor, clinician, or other medical professional.

The infectious disease diagnostic test can detect pathogens that of immediate public health concern. In some cases, a report may indicate that a sample is associated with one or more such pathogens. Accordingly, the application may use visual and/or textual cues for reporting Critical Alerts regarding public health pathogens. For example, the application may indicate that a pathogen of public health concern is present in a patient sample, and users may subsequently quarantine the patient or institute other protocols to prevent the pathogen from transferring to other persons or materials.

In some embodiments, the web-based application may provide a user with a diagnostic test profile. A diagnostic test profile may provide one or more properties associated with a subset of organisms within a scope of a diagnostic test. In some cases, the one or more properties comprises an organism name, an organism taxonomic rank, an organism class type, an organism sub-class, the organism membership in group based on phylogenetic and/or semantic relationship, medical relevance of an organism, validation, pathogen, RNA sensitive cutoff percentage, RNA specific cutoff percentage, DNA sensitive cutoff percentage, DNA specific cutoff percentage, highest scoring kmer, quantity of a particular kmer, or a combination thereof. In some cases, pathogen, organism taxonomic rank or organism class types may be as described elsewhere herein.

In some cases, medically relevant may be whether an organism may be associated with any disease. In some cases, medically relevant may be whether an organism is mentioned within a publication. In some cases, medically relevant may be whether an organism name is within a publication. In some cases, medically relevant may be displayed on the diagnostic test profile. In some cases, medically relevant may be indicated by a flag (yes/no) based on a threshold of relevance. The threshold of relevance may be dependent on the number of publications that organism may be mentioned within.

In some cases, validation may refer to in-silico validation. In some cases, validation may refer to in-silico validation where sequences from known public sequence repositories may be added as simulated sequencing reads into background reads from sequencing non-pathogen containing (negative) samples.

In some cases, the diagnostic test profile may provide a user with a narrower scope of organisms as procured by the methods and systems described elsewhere herein. In some cases, the scope of organisms may be any organism. In some cases, the scope of organisms may be taken from the reference databases described elsewhere herein. In some cases, the user may expand the set of organisms. In some cases, the user may narrow the set of organisms. The user may expand the set of organisms to view unexpected organisms. The user may narrow the set of organisms to view more relevant organisms.

In some embodiments, the diagnostic test profile may display and/or calculate properties associated with a subset of organisms within the scope of organisms from the diagnostic test. The diagnostic test profile may display and/or calculate at least about 1, 2, 3, 4, 5, 6, 7, 8, 9, 10, 15, 20, 25, 30, 35, 40, 45, 50, 75, 100, 500, 1000, 5000, or more properties. The diagnostic test profile may display and/or calculate at most about 5000, 1000, 500, 100, 75, 50, 45, 40, 35, 30, 25, 20, 15, 10, 9, 8, 7, 6, 5, 4, 3, 2 or less properties. The diagnostic test profile may display and/or calculate 1 to 5000, 1 to 1000, 1 to 500, 1 to 50, 1 to 25, 1 to 10, 1 to 5, or 1 to 3 properties. In some cases, the properties may be selected by a user and/or computer. In some cases, the properties may be pre-selected by a user and/or computer.

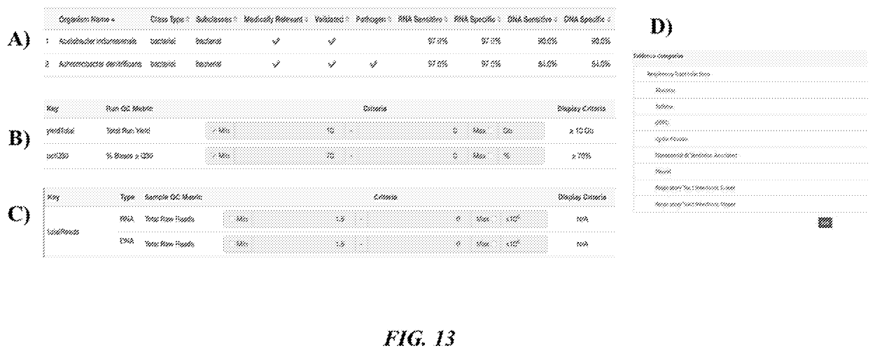

FIG. 13A shows an exemplary visualization for the diagnostic test profile. The visualization shows an organism name, class type of the organism, subclasses of the organism, binary illustration of medically relevant (green check mark may indicate medically relevant, lack of a green check mark may indicate not validated), binary illustration validated (green check mark may indicate validated, lack of a green check mark may indicate not validated), binary illustration of pathogen (green check mark may indicate medically relevant, lack of a green check mark may indicate not validated), RNA sensitive cutoff values, RNA specific cutoff values, DNA sensitive cutoff values, and DNA specific cutoff values. The visualization shows two rows of data pertaining to a diagnostic test profile. The visualization shows two rows of data with different organism names.

In some embodiments, the visualization may be displayed as a table with rows and columns. In some cases, the visualization may be displayed as a list, graph, chart, venn diagram, or numeric indicators, etc. In some cases, the visualization may be adjusted by the user or a computer. In some cases, the visualization may be adjusted to a specific format tailored to the desire or need of a user.

In some embodiments, the properties displayed by the visualization may be, for example, organism names, organism taxonomic ranks, organism class types, organism sub-class types, pathogens, RNA sensitive cutoff percentage, RNA specific cutoff percentage, DNA sensitive cutoff percentage, DNA specific cutoff percentage, medically relevant, and validated, etc. In some cases, the diagnostic test profile may have at least about 1, 2, 3, 4, 5, 6, 7, 8, 9, 10, 15, 20, 30, 40, 50, 100, 500, 1000 or more rows of data pertaining to a diagnostic test profile. In some cases, the diagnostic test profile may have at most about 1000, 500, 100, 50, 40, 30, 20, 15, 10, 9, 8, 7, 6, 5, 4, 3, 2, or less rows of data pertaining to a diagnostic test profile. In some cases, the diagnostic test profile may have from about 1 to 1000, 1 to 100, 1 to 50, 1 to 10, or 1 to 5 rows of data pertaining to a diagnostic test profile.

In some cases, the RNA sensitive cutoff percentage displayed and/or selected may be at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more. In some cases, the RNA sensitive cutoff percentage may be at most about 100%, 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, 60%, 55%, 50% or less. In some cases, the RNA sensitive cutoff percentages may be from about 50% to 100%, 60% to 100%, 70% to 100%, 80% to 100%, 85% to 100%, 90% to 100%, or 95% to 100%.

In some cases, the RNA specific cutoff percentage displayed and/or selected may be at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more. In some cases, the RNA specific cutoff percentage may be at most about 100%, 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, 60%, 55%, 50% or less. In some cases, the RNA specific cutoff percentage may be from about 50% to 100%, 60% to 100%, 70% to 100%, 80% to 100%, 85% to 100%, 90% to 100%, or 95% to 100%.

In some cases, the DNA sensitive cutoff percentage displayed and/or selected may be at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more. In some cases, the DNA sensitive cutoff percentage may be at most about 100%, 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, 60%, 55%, 50% or less. In some cases, the DNA sensitive cutoff percentage may be from about 50% to 100%, 60% to 100%, 70% to 100%, 80% to 100%, 85% to 100%, 90% to 100%, or 95% to 100%.

In some cases, the DNA specific cutoff percentage displayed and/or selected may be at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more. In some cases, the DNA specific cutoff percentage may be at most about 100%, 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, 60%, 55%, 50% or less. In some cases, the DNA specific cutoff percentage may be from about 50% to 100%, 60% to 100%, 70% to 100%, 80% to 100%, 85% to 100%, 90% to 100%, or 95% to 100%.

In some embodiments, the diagnostic test profile may display and/or calculate the run-level quality control criteria for the diagnostic test. FIG. 13B shows an exemplary visualization for the run-level quality control. The run-level quality control visualization shows a key, run quality control metric, criteria, display criteria, yield total, percentage of Q30, percentages of bases with greater than Q30, display criteria percentages, and display criteria data size. The run-level quality control visualization shows two rows of data pertaining to the run-level quality control information. The run-level quality control visualization shows that the criteria has a minimum that may be selected or unselected. The run-level quality control visualization shows that the criteria has a maximum that may be selected or unselected. The run-level quality control visualization shows that the criteria has values that a user or computer may input or adjust.

In some embodiments, the run-level quality control visualization may have at least about 1, 2, 3, 4, 5, 6, 7, 8, 9, 10, 15, 20, 30, 40, 50, 100, 500, 1000 or more rows of data pertaining to the run-level quality control. In some cases, the run-level quality control visualization may have at most about 1000, 500, 100, 50, 40, 30, 20, 15, 10, 9, 8, 7, 6, 5, 4, 3, 2, or less rows of data pertaining to the run-level quality control. In some cases, the run-level quality control visualization may have from about 1 to 1000, 1 to 100, 1 to 50, 1 to 10, or 1 to 5 rows of data pertaining to the run-level quality control.

In some embodiments, the run-level quality control visualization may be displayed as a table with rows and columns. In some cases, the run-level quality control visualization may be displayed as a list, graph, chart, venn diagram, or numeric indicators, etc. In some cases, the run-level quality control visualization may be adjusted by the user or a computer. In some cases, the run-level quality control visualization may be adjusted to a specific format tailored to the desire or need of a user.

In some embodiments, the run-level metrics may be, for example, total yield, total run yield, yield perfect, percentage of bases greater than or equal to Q30 (% Q>=30), cluster density, percentage of clusters passing filter, PhiX error rate, percentage of tile pass, intensity of A, intensity of C, projected total yield, yield <=n errors, etc.

In some cases, total yield may be the number of bases sequenced. In some cases, the total yield may be updated as the run progresses.

In some cases, total run yield may be the number of bases sequenced. In some cases, total run yield may be the number of bases sequenced which passed filter.

In some cases, yield perfect may be the number of bases in reads that align perfectly. In some cases, yield perfect may be the number of baes in reads that align perfectly as determined by alignment to PhiX of reads derived from a spiked in PhiX control sample. In some cases, if a PhiX control sample is not run in the lane, this chart may not be available.

In some cases, % Q>=30 may be the percentage of bases with a quality score of 30 or higher. In some cases, the chart may be generated after the 25th cycle. In some cases, the values represent the current cycle.

In some cases, cluster density may be the density of clusters (in thousands per mm²) detected by image analysis. In some cases, cluster density may be the density of clusters (in thousands per mm²) detected by image analysis, +/−one standard deviation.

In some cases, percentage of clusters passing filter may be the percentage of clusters passing filtering, +/−one standard deviation.

In some cases, PhiX error rate may be the calculated error rate, as determined by a spiked in PhiX control sample.

In some cases, percentage of tile pass may be the percentage of tiles that have a passing value. In some cases, the tile may indicate the progress of base calling. In some cases, the tile may indicate the quality scoring.

In some cases, intensity of A may be the average of the A channel intensity measured at the first cycle averaged over filtered clusters. In some cases, intensity of A may be the A channel intensity.

In some cases, intensity of C may be the average of the C channel intensity measured at the first cycle averaged over filtered clusters. In some cases, intensity of C may be the C channel intensity.

In some cases, projected total yield may be the projected number of bases expected to be sequenced at the end of the run.

In some cases, yield <=n errors may be the number of bases in reads that align with n errors or less, as determined by a spiked in PhiX control sample. N may be any integer, for example, 1, 2, 3, 4, 5, 6, 7, 8, 9, 10, etc.

In some embodiments, the diagnostic test profile may display and/or calculate the sample-level quality control criteria for the diagnostic test. FIG. 13C shows an exemplary visualization for the sample-level quality control. The sample-level quality control visualization shows a key, type, sample quality control metric, criteria, display criteria, total reads, RNA type, DNA type, and total raw reads. The sample-level quality control visualization shows two rows of data pertaining to the run-level quality control information. The sample-level quality control visualization shows that the criteria has a minimum that may be selected or unselected. The sample-level quality control visualization shows that the criteria has a maximum that may be selected or unselected. The sample-level quality control visualization shows that the criteria has values that a user or computer may input or adjust.

In some embodiments, the sample-level quality control visualization may have at least about 1, 2, 3, 4, 5, 6, 7, 8, 9, 10, 15, 20, 30, 40, 50, 100, 500, 1000 or more rows of data pertaining to the sample-level quality control. In some cases, the sample-level quality control visualization may have at most about 1000, 500, 100, 50, 40, 30, 20, 15, 10, 9, 8, 7, 6, 5, 4, 3, 2, or less rows of data pertaining to the sample-level quality control. In some cases, the sample-level quality control visualization may have from about 1 to 1000, 1 to 100, 1 to 50, 1 to 10, or 1 to 5 rows of data pertaining to the sample-level quality control.

In some embodiments, the run-level quality control visualization may be displayed as a table with rows and columns. In some cases, the run-level quality control visualization may be displayed as a list, graph, chart, venn diagram, or numeric indicators, etc. In some cases, the run-level quality control visualization may be adjusted by the user or a computer. In some cases, the run-level quality control visualization may be adjusted to a specific format tailored to the desire or need of a user.

In some embodiments, the sample-level metrics may be, for example, total raw reads, unique reads, post-adaptor reads, post-quality reads, total IC norm reads, entropy, G content, library Q score, library size, library concentration, etc.

In some cases, raw reads may be the reads in a file. In some cases, raw reads may be reads in a demultiplexed Fastq file.

In some cases, unique reads may be unique reads in a file. In some cases, unique reads may be unique reads in a demultiplexed Fastq file.

In some cases, post-adaptor reads may be reads after adaptor trimming in a file. In some cases, post-adaptor reads may be reads after adaptor trimming of a demultiplexed Fastq file.

In some cases, post-quality reads may be reads after applying a quality filter and trimming. In some cases, post-quality reads may be reads after applying a quality filter. In some cases, post-quality reads may be reads after applying trimming.

In some cases, total IC norm reads may be normalized read count of internal control organism(s).

In some cases, entropy may be the Shannon Diversity index of sequence complexity in the post-quality Fastq.

In some cases, library Q score may be the Phred scaled quality score of base calls in the post-quality Fastq.

In some cases, library size may be the estimate library size based on electrophoresis. In some cases, library size may be the estimate library size based on electrophoresis in the lab.

In some cases, library concentration may be the estimated library concentration based on qPCR or other methods. In some cases, library concentration may be the estimated library concentration based on qPCR in the lab.

In some embodiments, the properties, run-level criteria, and/or sample-level criteria may be tuned by a user through a graphical interface as shown in FIG. 13A-C. In some cases, the properties, run-level criteria, and/or sample-level criteria may be tuned by a computer and/or a user. In some cases, the amount of properties, run-level criteria, and/or sample-level criteria displayed may be reduced. In some cases, the amount of properties, run-level criteria, and/or sample-level criteria may be increased.

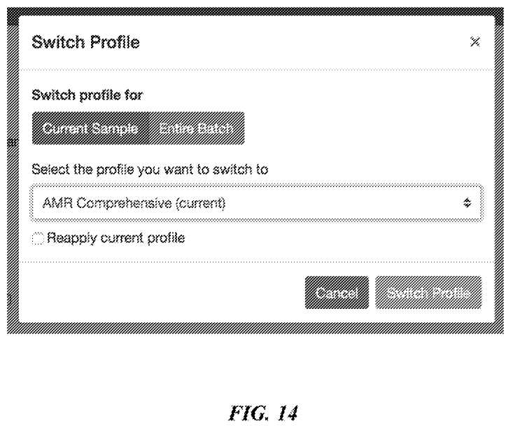

In some embodiments, a user may change the diagnostic test profile that is displayed. A user may change a diagnostic test profile to expand the set of organisms to look for unexpected organisms or to narrow the set for more relevant organisms. FIG. 14 shows an exemplary visualization for switching diagnostic test profiles. The switching diagnostic test profile visualization shows different batches which have different names. The switching diagnostic test profile visualization has a drop-down menu that a user can use to switch profiles. The switching diagnostic test visualization has an option to cancel switching profiles as well as the option to switch profiles. The switching diagnostic test visualization has the option to reapply the current profile.

In some cases, the user may view more than a single diagnostic test profile. In some cases, the user may view at least about 1, 2, 3, 4, 5, 6, 7, 8, 9, 10, 15, 20, 25, 30, 35, 40, 45, 50, 100, 500, 1000 or more diagnostic test profiles. In some cases, the user may view at most about 1000, 500, 100, 50, 45, 40, 35, 30, 25, 20, 15, 10, 9, 8, 7, 6, 5, 4, 3, 2 or less diagnostic test profiles. In some cases, the user may view about 1 to 1000, 1 to 100, 1 to 50, 1 to 10, or 1 to 5 diagnostic profiles. In some cases, the user may combine diagnostic test profiles. In some cases, the user may generate a report of one or more diagnostic test profiles. In some cases, the user may save a diagnostic test profile. In some cases, the user may give a diagnostic test profile a name. In some cases, the name of a diagnostic test profile may be randomly generated. In some cases, the diagnostic test profile may be used as a template for a different diagnostic template. In some cases, the user may select a different profile using, for example, a drop-down menu of profiles, a list of profiles, or a row of profiles, etc. The user may have at least about 1, 2, 3, 4, 5, 6, 7, 8, 9, 10, 11, 12, 13, 14, 15, 20, 30, 40, 50, 100, 500, 1000 or more saved diagnostic test profiles. The user may have at most about 1000, 500, 100, 50, 40, 30, 20, 15, 14, 13, 12, 11, 10, 9, 8, 7, 6, 5, 4, 3, 2, or less saved diagnostic test profiles. The user may have from about 1 to 1000, 1 to 100, 1 to 10, or 1 to 5 saved diagnostic test profiles.

In some embodiments, the diagnostic test profile may apply a disease category. The disease category may limit the scope of diagnostic test results. In some cases, the user may further limit the scope by selecting a disease sub-category as shown in FIG. 13D. The visualization shown in FIG. 13D displays a disease category. The visualization shows sub-categories of the disease. The disease category and disease sub-categories are shown in a drop-down menu and can be selected by a user. A disease category may be any disease, for example, respiratory tract infection. A disease sub-category may be any disease. A disease sub-category may be any disease that is within the scope of a larger disease category, for example, asthma falls under the scope of respiratory tract infections. In some cases, a user may define their own disease categories and/or disease sub-categories. In some cases, the disease category may be given a name. In some cases, the user may select the disease and/or disease sub-category using, for example, a drop-down menu, graph, search box, list, or chart, etc.

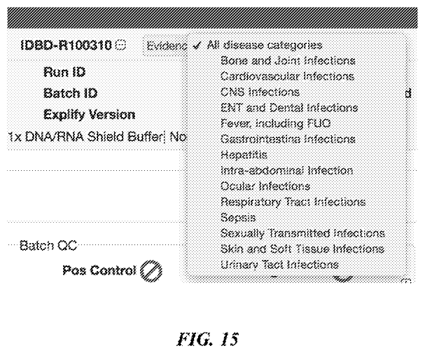

In some embodiments, the web-based application may provide more information of the organisms. The web-based application may provide a user with a collection of information. In some cases, the collection of information may be displayed on a diagnostic test profile. The collection of information may be, for example, publications (e.g. scientific publications, news publications, etc). The publications may associate an organism with disease categories. The disease categories may be any disease. The disease categories may be, for example, bone and join infections, cardiovascular infections, central nervous system (CNS) infections, enteric nervous system (ENT) and dental infections, fever including fevers of unknown origin (FUO), gastrointestinal infections, hepatitis, intra-abdominal infection, ocular infections, etc. FIG. 15 shows an exemplary visualization that may allow a user to select a disease category using a graphical user interface. The visualization shows a drop-down menu with the disease categories that a user can select. The selection of a disease category can narrow the search results to organisms that pertain to that disease category. The visualization also displays the run identification and the batch identification numbers of the diagnostic test. The visualization also shows the current version of software. The visualization can show one or more disease or disease sub-categories. The user may narrow the disease or disease sub-categories so that a selection can be viewed. In some cases, the user may select the disease and/or disease sub-category using, for example, a drop-down menu, graph, search box, list, or chart, etc. The visualization can show any other information to a user.

In some embodiments, the collection of information may be categorized by a user and/or computer. The collection of information may be categorized by a natural language processing system. The natural language processing system may be trained by a user and/or computer. The natural language processing system may have a user and/or computer set parameters. The parameters may be, for example syntax, semantics, discourse, or speech style, etc. The collection of information may be categorized on certain keywords found in the publications, potential pathogens associated with a disease, a user's understanding of the field, etc. The natural language processing system may be updated at any time. In some cases, the collection of information may be given a name, for example, evidence.

In some cases, when a category is selected by the user, the collection of information may be presented by an external source outside the web-based application. In some cases, the collection of information may be presented to the user within the web-based application. In some cases, the collection of information may be from a web search engine, for example, Google, Bing, or Yahoo, etc. In some cases, the collection of information may be from a database, for example, NCBI PubMed, PubMed, Scifinder, or Google Scholar, etc. In some cases, the database and/or web search engine may present to a user a list of publications.

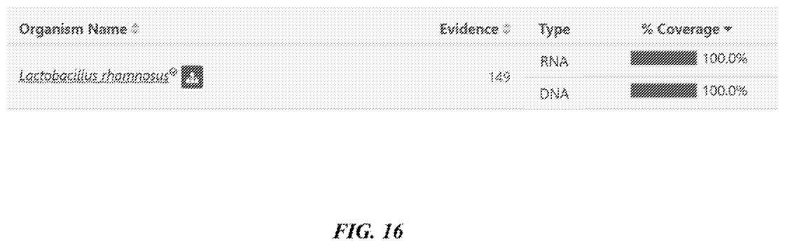

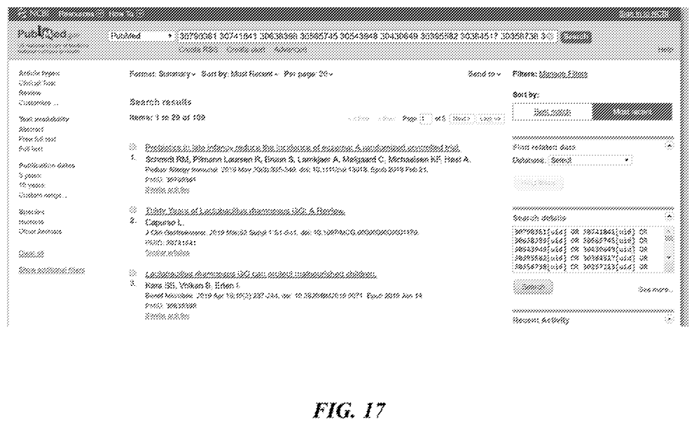

In some embodiments, one or more publications may be displayed on the diagnostic test profile as shown in FIG. 16. In FIG. 16, the visualization shows the organism name, Lacobacillus rhamnosus next to a clickable icon that can link a user to the phylogenetic tree. In addition, the visualization shows the number of publications (e.g. 149) that pertain to the organism name. The visualization also shows the type and percentage coverage. The percentage coverage has a numerical and color indicator. The number of publications may be an indirect measurement of relevance. In some cases, the organisms may be sorted by the number of publications. In some cases, the number of publications may be a hyperlink that may send a user to a webpage and/or database that may display each publication to the user, as shown in FIG. 17. As shown in FIG. 17, a list of publications that pertain to the Lactobacillus rhamnosus are displayed. When the user clicks on the number of publications, the user is sent to an external website. The publications are displayed by PubMed website. The selection of publications displayed have been procured beforehand. The selection of publications may be procured by a user or computer. The selection of publications may be procured on relevance. Relevance may have a variety of criteria that a user or computer may define beforehand or after.

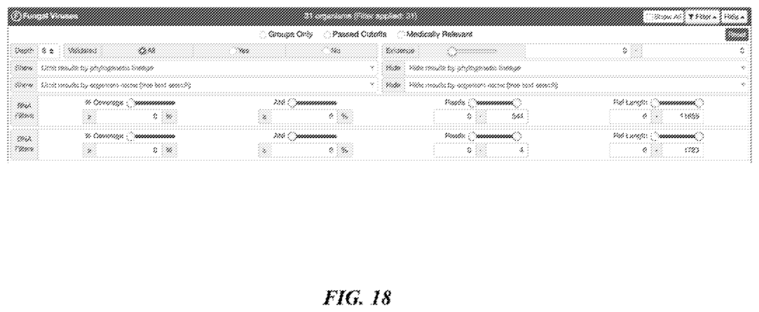

In some embodiments, the user may apply a filter to the diagnostic test profile. The user may apply a filter to refine or expand the set of detected organisms. The user may apply a filter to avoid false negative results. FIG. 18 shows an exemplary visualization of a filter interface that a user may use. The filter interface visualization shows a variety of filters that a user can use to expand or narrow the results from the diagnostic test. For example, the filter interface visualization shows that a user can: limit/expand by the percentage coverage using the slider icon or inputting a value of the RNA filter, limit/expand by the average nucleotide identity using the slider icon or inputting a value of the RNA filter, limit/expand by the reads using the slider icon or inputting a value of the RNA filter, limit/expand by the reference length using the slider icon or inputting a value of the RNA filter, limit/expand by the percentage coverage using the slider icon or inputting a value of the DNA filter, limit/expand by the average nucleotide identity using the slider icon or inputting a value of the DNA filter, limit/expand by the reads using the slider icon or inputting a value of the DNA filter, limit/expand by the reference length using the slider icon or inputting a value of the DNA filter. The filter interface visualization also shows that a user can limit/expand results by phylogenetic lineage, limit/expand results by organism name by free text search, hide results by phylogenetic lineage, hide results by organism name using free text search, limit/expand by the quantity of evidence.

In some cases, the RNA filter coverage percentage coverage may be at least about 0%, 5%, 10%, 15%, 20%, 25%, 30%, 35%, 40%, 45%, 50%, 55%, 60%, 65%, 70%, 75%, 80%, 85%, 90%, 95%, 99% or more. The RNA filter coverage percentage coverage may be at most about 99%, 95%, 90%, 85%, 80%, 75%, 70%, 65%, 60%, 55%, 50%, 45%, 40%, 35%, 30%, 25%, 20%, 15%, 10%, 5%, or less. The RNA filter coverage percentage coverage may be from about 0% to 100%, 0% to 95%, 0% to 90%, 0% to 85%, 0% to 80%, 0% to 75%, 0% to 70%, 0% to 65%, 0% to 60%, 0% to 55%, 0% to 50%, 0% to 45%, 0% to 40%, 0% to 35%, 0% to 30%, 0% to 25%, 0% to 20%, 0% to 15%, 0% to 10%, or 0% to 5%.

In some cases, the RNA filter average nucleotide identity may be at least about 0%, 5%, 10%, 15%, 20%, 25%, 30%, 35%, 40%, 45%, 50%, 55%, 60%, 65%, 70%, 75%, 80%, 85%, 90%, 95%, 99% or more. The RNA filter average nucleotide identity may be at most about 99%, 95%, 90%, 85%, 80%, 75%, 70%, 65%, 60%, 55%, 50%, 45%, 40%, 35%, 30%, 25%, 20%, 15%, 10%, 5%, or less. The RNA filter average nucleotide identity may be from about 0% to 100%, 0% to 95%, 0% to 90%, 0% to 85%, 0% to 80%, 0% to 75%, 0% to 70%, 0% to 65%, 0% to 60%, 0% to 55%, 0% to 50%, 0% to 45%, 0% to 40%, 0% to 35%, 0% to 30%, 0% to 25%, 0% to 20%, 0% to 15%, 0% to 10%, or 0% to 5%.

In some cases, the RNA filter reads may be at least about 0, 5, 10, 15, 30, 50, 100, 200, 300, 400, 500, 600, 700, 800, 900, 1000, 10000 or more. The RNA filter reads may be at most about 10000, 1000, 900, 800, 700, 600, 500, 400, 300, 200, 100, 50, 30, 15, 10, 5 or less. The RNA filter reads may be from about 0 to 10000, 0 to 1000, 0 to 500, 0 to 100, 0 to 50, or 0 to 5.

In some cases, the RNA filter reference length may be at least about 0, 5, 10, 15, 30, 50, 100, 200, 300, 400, 500, 600, 700, 800, 900, 1000, 10000, 20000, 50000 or more. The RNA filter reads may be at most about 50000, 20000, 10000, 1000, 900, 800, 700, 600, 500, 400, 300, 200, 100, 50, 30, 15, 10, 5 or less. The RNA filter reads may be from about 0 to 50000, 0 to 20000, 0 to 10000, 0 to 1000, 0 to 500, 0 to 100, 0 to 50, or 0 to 5.

In some cases, the DNA filter coverage percentage coverage may be at least about 0%, 5%, 10%, 15%, 20%, 25%, 30%, 35%, 40%, 45%, 50%, 55%, 60%, 65%, 70%, 75%, 80%, 85%, 90%, 95%, 99% or more. The DNA filter coverage percentage coverage may be at most about 99%, 95%, 90%, 85%, 80%, 75%, 70%, 65%, 60%, 55%, 50%, 45%, 40%, 35%, 30%, 25%, 20%, 15%, 10%, 5%, or less. The DNA filter coverage percentage coverage may be from about 0% to 100%, 0% to 95%, 0% to 90%, 0% to 85%, 0% to 80%, 0% to 75%, 0% to 70%, 0% to 65%, 0% to 60%, 0% to 55%, 0% to 50%, 0% to 45%, 0% to 40%, 0% to 35%, 0% to 30%, 0% to 25%, 0% to 20%, 0% to 15%, 0% to 10%, or 0% to 5%.

In some cases, the DNA filter average nucleotide identity may be at least about 0%, 5%, 10%, 15%, 20%, 25%, 30%, 35%, 40%, 45%, 50%, 55%, 60%, 65%, 70%, 75%, 80%, 85%, 90%, 95%, 99% or more. The DNA filter average nucleotide identity may be at most about 99%, 95%, 90%, 85%, 80%, 75%, 70%, 65%, 60%, 55%, 50%, 45%, 40%, 35%, 30%, 25%, 20%, 15%, 10%, 5%, or less. The DNA filter average nucleotide identity may be from about 0% to 100%, 0% to 95%, 0% to 90%, 0% to 85%, 0% to 80%, 0% to 75%, 0% to 70%, 0% to 65%, 0% to 60%, 0% to 55%, 0% to 50%, 0% to 45%, 0% to 40%, 0% to 35%, 0% to 30%, 0% to 25%, 0% to 20%, 0% to 15%, 0% to 10%, or 0% to 5%.

In some cases, the DNA filter reads may be at least about 0, 5, 10, 15, 30, 50, 100, 200, 300, 400, 500, 600, 700, 800, 900, 1000, 10000 or more. The DNA filter reads may be at most about 10000, 1000, 900, 800, 700, 600, 500, 400, 300, 200, 100, 50, 30, 15, 10, 5 or less. The DNA filter reads may be from about 0 to 10000, 0 to 1000, 0 to 500, 0 to 100, 0 to 50, or 0 to 5.

In some cases, the DNA filter reference length may be at least about 0, 5, 10, 15, 30, 50, 100, 200, 300, 400, 500, 600, 700, 800, 900, 1000, 10000, 20000, 50000 or more. The DNA filter reads may be at most about 50000, 20000, 10000, 1000, 900, 800, 700, 600, 500, 400, 300, 200, 100, 50, 30, 15, 10, 5 or less. The DNA filter reads may be from about 0 to 50000, 0 to 20000, 0 to 10000, 0 to 1000, 0 to 500, 0 to 100, 0 to 50, or 0 to 5.

In some embodiments, the filters may be adjusted using a graphical user interface. The filter may be, for example, organism characteristics. Organism characteristics may be, for example, validation status, number of publications, membership in groups, phylogenetic linear, taxonomy, kmer count, or a combination thereof. In some cases, the user may filter using a word and/or text search. In some cases, a filter may be based on artificial intelligence (AI). In some cases, the AI may learn from previous data. In some cases, the AI may report an organism that it classifies as most relevant. In some cases, a filter may be based on a machine learning algorithm. The machine learning algorithm may comprise a deep neural network. The machine learning algorithm may comprise a convolutional neural network.

In some embodiments, the diagnostic test profile may have at least about 1, 2, 3, 4, 5, 6, 7, 8, 9, 10, 15, 20, 25, 30, 35, 40, 45, 50, 100, 500, 1000 or more filters. In some cases, the diagnostic test profile may have at most about 1000, 500, 100, 50, 45, 40, 35, 30, 25, 20, 15, 10, 9, 8, 7, 6, 5, 4, 3, 2 or less filters. In some cases, the diagnostic test profile may have 1 to 1000, 1 to 100, 1 to 50, 1 to 10, or 1 to 5 filters.

In some embodiments, the user may adjust the filter at any point in time during data processing. In some cases, the filters are pre-selected by a user and/or computer. In some cases, the filters may be used for more than one diagnostic profile. In some cases, the diagnostic test profile may have the same filters as a different test profile. In some cases, the diagnostic test profile may have different filters than a different test profile.

In some embodiments, the user may fine-tune criteria for the filters. The criteria may be from the diagnostic test. The criteria may be based on intermediate organism classification results. The criteria may be results from RNA and/or DNA sequences. The criteria may be, for example, the percentage coverage, average nucleotide identity, sequence reads, reference length, or as described elsewhere herein, etc. In some case, the filters may apply a range of values for the criteria. The user may set a range for the criteria. A computer may set the range for the criteria. The range may be any value.

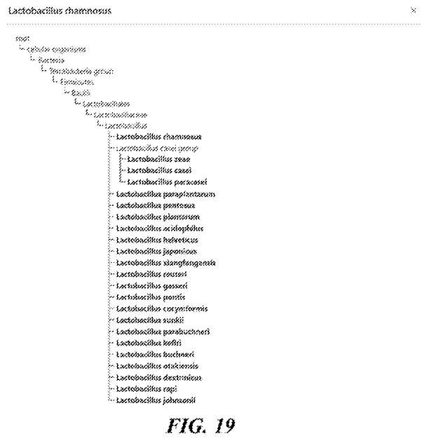

In some embodiments, the web-based application may display to a user one or more results of organism classification. In some cases, the organisms may be unclassified. The organisms may be classified as groups of phylogenetically related organisms. FIG. 19 shows exemplary visualization of classifying organisms. The visualization of the classified organism shows the different members of the phylogenetic tree. The phylogenetic tree shows the possibilities of classes the organism may be from. The class at the top is the one that the software prescribes as the most likely depending on a set of criteria as described elsewhere herein.

In some cases, the members of the classified organisms may be sorted. The member may be sorted depending on criteria, for example, percentage of coverage RNA, percentage of coverage DNA, average nucleotide identity for RNA, average nucleotide identity for DNA, read counts for RNA, or read counts for DNA, or number of relevant publications, etc. In some cases, the sorting may depend on at least about 1, 2, 3, 4, 5, 6, 7, 8, 9, 10, or more criteria. In some cases, the sorting may depend on at most about 10, 9, 8, 7, 6, 5, 4, 3, 2, or less criteria. In some cases, the sorting may depend on 1 to 10, 1 to 8, 1 to 6, 1 to 4, or 1 to 3 criteria.

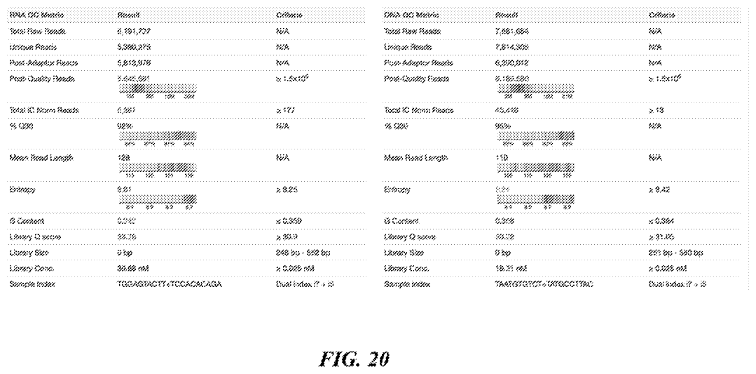

In some embodiments, the web-based application may display to a user quality control metrics as shown in FIG. 20. The metrics may be, for example, total raw reads, unique reads, post-adaptor reads, post-quality reads, total IC norm reads, percentage of bases with a quality score of 30 or higher (% Q30), mean read length, entropy, G Content, library Q score, library size, library concentration, sample index, mean read length, etc. The metrics may be as described elsewhere herein. The metrics may be for RNA metrics and/or DNA metrics. In some cases, the metrics may be displayed. In some cases, the metrics may display a value or number. In some cases, the metrics may be displayed in chart, for example, a horizontal bar chart, vertical bar chart, pie chart, venn diagram. In some cases, the display may display at least about 1, 2, 3, 4, 5, 6, 7, 8, 9, 10, 11, 12, 13, 14, 15, 16, 17, 18, 19, 20, 21, 22, 23, 24, 25, 50, 100, 500, or more metrics. In some cases, the display may display at most about 500, 100, 50, 25, 24, 23, 22, 21, 20, 19, 18, 17, 16, 15, 14, 13, 12, 11, 10, 9, 8, 7, 6, 5, 4, 3, 2, or less metrics. In some cases, the display may display 1 to 500, 1 to 100, 1 to 50, 1 to 25, 1 to 10, or 1 to 5 metrics.

In some cases, mean read length may be after adaptor and quality trimming the reads in the Fastq. In some cases, the reads in the Fastq may be less than in the original demultiplexed Fastq. In some cases, the mean of the shortened reads may give an indication of the extent of trimming.

In some cases, sample index(es) may be the nucleotides (ntd) added to the sequencing libraries that may enable multiplexed sequencing (many sample libraries on one flowcell). In some cases, the number of nucleotides added may be at least about 1, 2, 3, 4, 5, 6, 7, 8, 9, 10, 11, 12, 13, 14, 15 or more. In some cases, the number of nucleotides added may be at most about 15, 14, 13, 12, 11, 10, 9, 8, 7, 6, 5, 4, 3, 2 or less. In some cases, the number of nucleotides added may be from about 1 to 15, 1 to 10, 1 to 5, 3 to 15, 3 to 12, 3 to 10, 3 to 5, 6 to 15, 6 to 12, or 6 to 10. In some cases, the index reads may provide the mechanism to de-multiplex the reads into separate Fastq files.

### Computer Systems

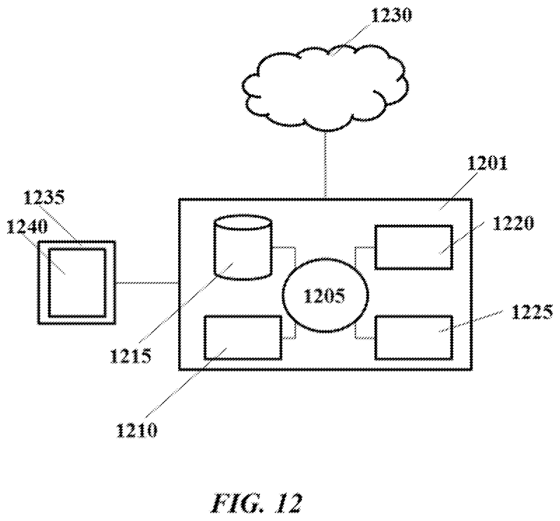

The present disclosure provides computer systems that are programmed to implement methods of the disclosure. FIG. 12 shows a computer system 1201 that is programmed or otherwise configured to process and/or assay a sample. The computer system 1201 may regulate various aspects of sample processing and assaying of the present disclosure, such as, for example, activation of a valve or pump to transfer a reagent or sample from one chamber to another or application of heat to a sample (e.g., during an amplification reaction). The computer system 1201 may be an electronic device of a user or a computer system that is remotely located with respect to the electronic device. The electronic device may be a mobile electronic device.

The computer system 1201 includes a central processing unit (CPU, also “processor” and “computer processor” herein) 1205, which may be a single core or multi core processor, or a plurality of processors for parallel processing. The computer system 1201 also includes memory or memory location 1210 (e.g., random-access memory, read-only memory, flash memory), electronic storage unit 1215 (e.g., hard disk), communication interface 1220 (e.g., network adapter) for communicating with one or more other systems, and peripheral devices 1225, such as cache, other memory, data storage and/or electronic display adapters. The memory 1210, storage unit 1215, interface 1220 and peripheral devices 1225 are in communication with the CPU 1205 through a communication bus (solid lines), such as a motherboard. The storage unit 1215 may be a data storage unit (or data repository) for storing data. The computer system 1201 may be operatively coupled to a computer network (“network”) 1230 with the aid of the communication interface 1220. The network 1230 may be the Internet, an internet and/or extranet, or an intranet and/or extranet that is in communication with the Internet. The network 1230 in some cases is a telecommunication and/or data network. The network 1230 may include one or more computer servers, which may enable distributed computing, such as cloud computing. The network 1230, in some cases with the aid of the computer system 1201, may implement a peer-to-peer network, which may enable devices coupled to the computer system 1201 to behave as a client or a server.

The CPU 1205 may execute a sequence of machine-readable instructions, which may be embodied in a program or software. The instructions may be stored in a memory location, such as the memory 1210. The instructions may be directed to the CPU 1205, which may subsequently program or otherwise configure the CPU 1205 to implement methods of the present disclosure. Examples of operations performed by the CPU 1205 may include fetch, decode, execute, and writeback.

The CPU 1205 may be part of a circuit, such as an integrated circuit. One or more other components of the system 1201 may be included in the circuit. In some cases, the circuit is an application specific integrated circuit (ASIC).

The storage unit 1215 may store files, such as drivers, libraries and saved programs. The storage unit 1215 may store user data, e.g., user preferences and user programs. The computer system 1201 in some cases may include one or more additional data storage units that are external to the computer system 1201, such as located on a remote server that is in communication with the computer system 1201 through an intranet or the Internet.

The computer system 1201 may communicate with one or more remote computer systems through the network 1230. For instance, the computer system 1201 may communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user may access the computer system 1201 via the network 1230.

Methods as described herein may be implemented by way of machine (e.g., computer processor) executable code stored on an electronic storage location of the computer system 1201, such as, for example, on the memory 1210 or electronic storage unit 1215. The machine executable or machine readable code may be provided in the form of software. During use, the code may be executed by the processor 1205. In some cases, the code may be retrieved from the storage unit 1215 and stored on the memory 1210 for ready access by the processor 1205. In some situations, the electronic storage unit 1215 may be precluded, and machine-executable instructions are stored on memory 1210.

The code may be pre-compiled and configured for use with a machine having a processer adapted to execute the code, or may be compiled during runtime. The code may be supplied in a programming language that may be selected to enable the code to execute in a pre-compiled or as-compiled fashion.

Aspects of the systems and methods provided herein, such as the computer system 1201, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media may include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1201 may include or be in communication with an electronic display 1235 that comprises a user interface (UI) 1240 for providing, for example, a current stage of processing or assaying of a sample (e.g., a particular operation, such as a lysis operation, that is being performed). Examples of UI's include, without limitation, a graphical user interface (GUI) and web-based user interface.

Methods and systems of the present disclosure may be implemented by way of one or more algorithms. An algorithm may be implemented by way of software upon execution by the central processing unit 1205.

Several aspects are described with reference to example applications for illustration. Unless otherwise indicated, any embodiment may be combined with any other embodiment. It should be understood that numerous specific details, relationships, and methods are set forth to provide a full understanding of the features described herein. A skilled artisan, however, will readily recognize that the features described herein may be practiced without one or more of the specific details or with other methods. The features described herein are not limited by the illustrated ordering of acts or events, as some acts may occur in different orders and/or concurrently with other acts or events. Furthermore, not all illustrated acts or events are required to implement a methodology in accordance with the features described herein.

Some inventive embodiments herein contemplate numerical ranges. When ranges are present, the ranges include the range endpoints. Additionally, every sub range and value within the range is present as if explicitly written out. The term “about” or “approximately” may mean within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Alternatively, particularly with respect to biological systems or processes, the term may mean within an order of magnitude, within 5-fold, or within 2-fold, of a value. Where particular values are described in the application and claims, unless otherwise stated the term “about” meaning within an acceptable error range for the particular value may be assumed.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-23. (canceled)

24. A computer-implemented method for providing a diagnostic test profile for determining whether a human is afflicted with a disease or disorder through exposure to a pathogen, the diagnostic test profile corresponding to a sample derived from the human, the method comprising:
(i) providing data corresponding to said sample, wherein said data comprises a plurality of sequencing reads;
(ii) providing an interface to a user, wherein said interface displays to said user
(a) an entity indicator indicating that said plurality of sequencing reads includes sequencing reads from a plurality of entities wherein said plurality entities comprises an entity that is a fungus, a bacterium, a parasite, or a virus, and wherein the entity indicator includes an organism name and organism type for each entity in the plurality of entities represented in the plurality of sequencing reads,
(b) an indication, for each respective entity in the plurality of entities, as to whether or not the respective entity is medically relevant based on whether or not the respective entity is mentioned in a threshold number of publications, and
(c) a sample quality control visualization quality control metric indicator indicating a quality of the plurality of sequencing reads in the form of (i) a user adjustable threshold minimum number of total raw RNA sequencing reads in the plurality of sequencing reads required to display said plurality of entities in the diagnostic test profile, and (ii) a user adjustable threshold minimum number of total raw DNA sequencing reads in the plurality of sequencing reads required to display said one or more entities in the diagnostic test profile; and
(iii) obtaining instructions to limit the diagnostic test profile to a disease category thereby determining whether the human is afflicted with a disease or disorder through exposure to a pathogen.

25. The computer-implemented method of claim 24, wherein said plurality of sequencing reads comprises deoxyribonucleic acid (DNA) sequencing reads and/or ribonucleic acid (RNA) sequencing reads.

26. The computer-implemented method of claim 25, wherein said plurality of sequencing reads comprises both DNA sequencing reads and RNA sequencing reads.

27. The computer-implemented method of claim 24, wherein said plurality of sequencing reads are generated using sequencing by synthesis, sequencing by ligation, nanopore sequencing, or sequencing by hybridization.

28. The computer-implemented method of claim 27, wherein said plurality of sequencing reads are generated using sequencing by synthesis.

29-32. (canceled)

33. The computer-implemented method of claim 24, wherein said second entity is associated with a disease or disorder.

34. The computer-implemented method of claim 24, wherein said second entity is associated with an infection.

35-36. (canceled)

37. The computer-implemented method of claim 24, wherein said human has or is suspected of having a disease or disorder.

38. The computer-implemented method of claim 24, wherein said human has been exposed or is suspected of having been exposed to a pathogen.

39-42. (canceled)

43. The computer-implemented method of claim 24, further comprising:
(i) performing with a computer system a sequence comparison between a sequencing read of said plurality of sequencing reads and a plurality of reference polynucleotide sequences, wherein the comparison comprises calculating k-mer weights as a measure of how likely it is that k-mers within said sequencing read are derived from a reference sequence within said plurality of reference polynucleotide sequences;
(ii) identifying said sequencing read as corresponding to a particular reference sequence in a database of reference sequences if the sum of k-mer weights for said reference sequence is above a threshold level; and
(iii) assembling a record database comprising reference sequences identified in (ii), wherein said record database excludes reference sequences to which no sequencing read corresponds.

44. The computer-implemented method of claim 24, further comprising:
(i) for each respective sequencing read of said plurality of sequencing reads:
(a) performing with a computer system a sequence comparison between the respective sequencing read and a plurality of reference polynucleotide sequences, wherein the comparison comprises calculating k-mer weights as a measure of how likely it is that k-mers within said respective sequencing read are derived from a reference sequence within said plurality of reference polynucleotide sequences; and
(b) calculating a probability that said respective sequencing read corresponds to a particular reference sequence in a database of reference sequences based on said k-mer weights, thereby generating a sequence probability;
(ii) calculating a score for the presence or absence of one or more taxa based on the sequence probabilities corresponding to sequences representative of said one or more taxa; and
(iii) identifying said one or more taxa as present or absent in said sample based on the corresponding scores.

45-81. (canceled)

82. The computer-implemented method of claim 24, wherein the disease or disorder is a bone and joint infection, a cardiovascular infection, a central nervous system (CNS) infection, an enteric nervous system (ENT) and dental infection, a gastrointestinal infection, hepatitis, an intra-abdominal infection, or an ocular infection.

83. The computer-implemented method of claim 24, wherein the sample is a bodily fluid.

84. The computer-implemented method of claim 24, wherein the bodily flue is blood, urine, saliva or sweat.

85. The computer-implemented method of claim 25, wherein said plurality of sequencing reads are generated using sequencing by synthesis, sequencing by ligation, nanopore sequencing, or sequencing by hybridization.

86. The computer-implemented method of claim 85, wherein said plurality of sequencing reads are generated using sequencing by synthesis.

87. A computer system for providing a diagnostic test profile for determining whether a human is afflicted with a disease or disorder through exposure to a pathogen, the diagnostic test profile corresponding to a sample derived from the human, the computer system comprising one or more processors, memory and one or more programs stored in the memory that, when executed by the one or more processors, cause the computer system to perform a method comprising:
(i) providing data corresponding to said sample, wherein said data comprises a plurality of sequencing reads;
(ii) providing an interface to a user, wherein said interface displays to said user
(a) an entity indicator indicating that said plurality of sequencing reads includes sequencing reads from a plurality of entities wherein said plurality entities comprises an entity that is a fungus, a bacterium, a parasite, or a virus, and wherein the entity indicator includes an organism name and organism type for each entity in the plurality of entities represented in the plurality of sequencing reads,
(b) an indication, for each respective entity in the plurality of entities, as to whether or not the respective entity is medically relevant based on whether or not the respective entity is mentioned in a threshold number of publications, and
(c) a sample quality control visualization quality control metric indicator indicating a quality of the plurality of sequencing reads in the form of (i) a user adjustable threshold minimum number of total raw RNA sequencing reads in the plurality of sequencing reads required to display said plurality of entities in the diagnostic test profile, and (ii) a user adjustable threshold minimum number of total raw DNA sequencing reads in the plurality of sequencing reads required to display said one or more entities in the diagnostic test profile; and
(iii) obtaining instructions to limit the diagnostic test profile to a disease category thereby determining whether the human is afflicted with a disease or disorder through exposure to a pathogen.

88. The computer system of claim 87, wherein the method further comprises:
(i) performing a sequence comparison between a sequencing read of said plurality of sequencing reads and a plurality of reference polynucleotide sequences, wherein the comparison comprises calculating k-mer weights as a measure of how likely it is that k-mers within said sequencing read are derived from a reference sequence within said plurality of reference polynucleotide sequences;
(ii) identifying said sequencing read as corresponding to a particular reference sequence in a database of reference sequences if the sum of k-mer weights for said reference sequence is above a threshold level; and
(iii) assembling a record database comprising reference sequences identified in (ii), wherein said record database excludes reference sequences to which no sequencing read corresponds.

89. The computer system of claim 87, wherein the method further comprises:
(i) for each respective sequencing read of said plurality of sequencing reads:
(a) performing with a sequence comparison between the respective sequencing read and a plurality of reference polynucleotide sequences, wherein the comparison comprises calculating k-mer weights as a measure of how likely it is that k-mers within said respective sequencing read are derived from a reference sequence within said plurality of reference polynucleotide sequences; and
(b) calculating a probability that said respective sequencing read corresponds to a particular reference sequence in a database of reference sequences based on said k-mer weights, thereby generating a sequence probability;
(ii) calculating a score for the presence or absence of one or more taxa based on the sequence probabilities corresponding to sequences representative of said one or more taxa; and
(iii) identifying said one or more taxa as present or absent in said sample based on the corresponding scores.

90. A non-transitory computer readable storage medium storing one or more programs that, when executed by one or more processors of a computer system, cause the computer system to perform a method for providing a diagnostic test profile for determining whether a human is afflicted with a disease or disorder through exposure to a pathogen, the diagnostic test profile corresponding to a sample derived from the human, the method comprising:
(i) providing data corresponding to said sample, wherein said data comprises a plurality of sequencing reads;
(ii) providing an interface to a user, wherein said interface displays to said user
(a) an entity indicator indicating that said plurality of sequencing reads includes sequencing reads from a plurality of entities wherein said plurality entities comprises an entity that is a fungus, a bacterium, a parasite, or a virus, and wherein the entity indicator includes an organism name and organism type for each entity in the plurality of entities represented in the plurality of sequencing reads,
(b) an indication, for each respective entity in the plurality of entities, as to whether or not the respective entity is medically relevant based on whether or not the respective entity is mentioned in a threshold number of publications, and
(c) a sample quality control visualization quality control metric indicator indicating a quality of the plurality of sequencing reads in the form of (i) a user adjustable threshold minimum number of total raw RNA sequencing reads in the plurality of sequencing reads required to display said plurality of entities in the diagnostic test profile, and (ii) a user adjustable threshold minimum number of total raw DNA sequencing reads in the plurality of sequencing reads required to display said one or more entities in the diagnostic test profile; and
(iii) obtaining instructions to limit the diagnostic test profile to a disease category thereby determining whether the human is afflicted with a disease or disorder through exposure to a pathogen.

91. The non-transitory computer readable storage medium of claim 90, wherein the method further comprises:
(i) performing a sequence comparison between a sequencing read of said plurality of sequencing reads and a plurality of reference polynucleotide sequences, wherein the comparison comprises calculating k-mer weights as a measure of how likely it is that k-mers within said sequencing read are derived from a reference sequence within said plurality of reference polynucleotide sequences;
(ii) identifying said sequencing read as corresponding to a particular reference sequence in a database of reference sequences if the sum of k-mer weights for said reference sequence is above a threshold level; and
(iii) assembling a record database comprising reference sequences identified in (ii), wherein said record database excludes reference sequences to which no sequencing read corresponds.

92. The non-transitory computer readable storage medium of claim 90, wherein the method further comprises:
(i) for each respective sequencing read of said plurality of sequencing reads:
(a) performing with a sequence comparison between the respective sequencing read and a plurality of reference polynucleotide sequences, wherein the comparison comprises calculating k-mer weights as a measure of how likely it is that k-mers within said respective sequencing read are derived from a reference sequence within said plurality of reference polynucleotide sequences; and
(b) calculating a probability that said respective sequencing read corresponds to a particular reference sequence in a database of reference sequences based on said k-mer weights, thereby generating a sequence probability;
(ii) calculating a score for the presence or absence of one or more taxa based on the sequence probabilities corresponding to sequences representative of said one or more taxa; and
(iii) identifying said one or more taxa as present or absent in said sample based on the corresponding scores.
